(19) United States
(12) Patent Application Publication
(10) Pub. No.: US 2008/0109925 A1
(43) Pub. Date: May 8, 2008

(54) Transcriptional Regulatory Nucleic Acids, Polypeptides and Methods of Use Thereof

(75) Inventors: Yumin Tao, Urbandale, IA (US); William J. Gordon-Kamm, Urbandale, IA (US); Bo Shen, Johnston, IA (US); Keith S. Lowe, Johnston, IA (US); Olga N. Danilevskaya, Johnston, IA (US); Pramod B. Mahajan, Urbandale, IA (US); Jan Antoni Rafalski, Wilmington, DE (US); Hajime Sakai, Newark, DE (US); Theodore M. Klein, Wilmington, DE (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL INC., Johnston, IA (US)

(21) Appl. No.: 11/779,552

(22) Filed: Jul. 18, 2007

Related U.S. Application Data
(63) Continuation of application No. 10/675,072, filed on Sep. 30, 2003, now abandoned.
Application No. 10/675,072 is a continuation-in-part of application No. 10/005,057, filed on Dec. 4, 2001, now abandoned.
(60) Provisional application No. 60/251,555, filed on Dec. 6, 2000.

(51) Int. Cl. A01H 5/00; C07H 21/04; C07K 16/00; C12N 15/00; C12P 21/04

(52) U.S. Cl. 800/306; 435/419; 435/468; 530/350; 536/23.6; 800/298; 800/312; 800/314; 800/320; 800/320.1; 800/320.2; 800/320.3; 800/322

(57) ABSTRACT

The invention provides isolated nucleic acids and their encoded proteins that act as cell transcription inhibitors and methods of use thereof. The invention further provides expression cassettes, transformed host cells, transgenic plants and plant parts, and antibody compositions.

### CROSS-REFERENCE TO RELATED APPLICATIONS

This application is a continuation of the non-provisional U.S. application Ser. No. 10/675,072 filed Sep. 30, 2003, and claims the benefit of non-provisional U.S. application Ser. No. 10/005,057 filed Dec. 4, 2001 and of provisional U.S. Application Ser. No. 60/251,555 filed Dec. 6, 2000, which applications are herein incorporated by reference.

### TECHNICAL FIELD

The present invention relates generally to plant molecular biology. More specifically, it relates to nucleic acids and methods for modulating their expression in plants.

### BACKGROUND OF THE INVENTION

Major advances in plant transformation have occurred over the last few years. However, in major crop plants, such as maize and soybeans, serious genotype limitations still exist. Transformation of agronomically important maize inbred lines continues to be both difficult and time consuming. Traditionally, the only way to elicit a culture response has been by optimizing medium components and/or explant material and source. This has led to success in some genotypes, but most elite hybrids fail to produce a favorable culture response. While, transformation of model genotypes is efficient, the process of introgressing transgenes into production inbreds is laborious, expensive and time consuming. It would save considerable time and money if genes could be introduced into and evaluated directly in production inbreds or commercial hybrids.

Current methods for genetic engineering in maize require a specific cell type as the recipient of foreign DNA. These cells are found in relatively undifferentiated, rapidly growing callus cells or on the scutellar surface of the immature embryo (which gives rise to callus). Irrespective of the delivery method currently used, DNA is introduced into literally thousands of cells, yet transformants are recovered at frequencies of 10⁻⁵ relative to transiently-expressing cells. Exacerbating this problem, the trauma that accompanies DNA introduction directs recipient cells into cell cycle arrest and accumulating evidence suggests that many of these cells are directed into apoptosis or programmed cell death. (Bowen, et al., Third International Congress of the International Society for Plant Molecular Biology, 1991, Abstract 1093). Therefore it would be desirable to provide improved methods capable of increasing transformation efficiency in a number of cell types.

Typically a selectable marker is used to recover transformed cells. Traditional selection schemes expose all cells to a phytotoxic agent and rely on the introduction of a resistance gene to recover transformants. Unfortunately, the presence of dying cells may reduce the efficiency of stable transformation. It would therefore be useful to provide a positive selection system for recovering transformants.

In spite of increases in yield and harvested area worldwide, it is predicted that over the next ten years, meeting the demand for corn will require an additional 20% increase over current production (Dowswell, C R, Paliwal, R L, Cantrell, R P, (1996) Maize in the Third World, Westview Press, Boulder, Colo.).

In hybrid crops, including grains, oil seeds, forages, fruits and vegetables, there are problems associated with the development and production of hybrid seeds. The process of cross-pollination of plants is laborious and expensive. In the cross-pollination process, the female plant must be prevented from being fertilized by its own pollen. Many methods have been developed over the years, such as detasseling in the case of corn, developing and maintaining male sterile lines, and developing plants that are incompatible with their own pollen, to name a few. Since hybrids do not breed true, the process must be repeated for the production of every hybrid seed lot.

To further complicate the process, inbred lines are crossed. For example in the case of corn, the inbreds can be low yielding. This provides a major challenge in the production of hybrid seed corn. In fact, certain hybrids cannot be commercialized at all due to the performance of the inbred lines. The production of hybrid seeds is consequently expensive, time consuming and provides known and unknown risks. It would therefore be valuable to develop new methods that contribute to the increase of production efficiency of hybrid seed.

As new traits are added to commercial crops by means of genetic engineering, problems arise in “stacking” traits. In order to develop heritable stacked traits, the traits must be linked because of segregating populations. Improved methods for developing hybrid seed that would not require linking of the traits would significantly shorten the time for developing commercial hybrid seeds.

Gene silencing is another problem in developing heritable traits with genetic engineering. Frequently gene silencing is seen following meiotic divisions. Elimination or reduction of this problem would advance the state of science and industry in this area.

### DETAILED DESCRIPTION OF THE INVENTION

### Definitions

The term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

As used herein, “nucleic acid” means a polynucleotide and includes single or double-stranded polymer of deoxyribonucleotide or ribonucleotide bases. Nucleic acids may also include fragments and modified nucleotides.

As used herein, “CHD polynucleotide” means a nucleic acid sequence encoding a CHD polypeptide. As used herein, “CHD polypeptide” means a polypeptide containing 3 domains, a chromatin organization modifier, a helicase SNF-2 related/ATP domain, and a DNA binding domain. CHD is an acronym based on the first letter of the names of the 3 domains.

As used herein, “polypeptide” means proteins, protein fragments, modified proteins, amino acid sequences and synthetic amino acid sequences. The polypeptide can be glycosylated or not.

As used herein, “plant” includes plants and plant parts including but not limited to plant cells, plant tissue such as leaves, stems, roots, flowers, and seeds.

As used herein, “promoter” includes reference to a region of DNA upstream from the start of transcription and involved in recognition and binding of RNA polymerase and other proteins to initiate transcription.

By “fragment” is intended a portion of the nucleotide sequence or a portion of the amino acid sequence and hence protein encoded thereby. Fragments of a nucleotide sequence may encode protein fragments that retain the biological activity of the native nucleic acid. Alternatively, fragments of a nucleotide sequence that are useful as hybridization probes may not encode fragment proteins retaining biological activity. Thus, fragments of a nucleotide sequence are generally greater than 25, 50, 100, 200, 300, 400, 500, 600 or 700 nucleotides and up to and including the entire nucleotide sequence encoding the proteins of the invention. Generally the probes are less than 1000 nucleotides and preferably less than 500 nucleotides. Fragments of the invention include antisense sequences used to decrease expression of the inventive polynucleotides. Such antisense fragments may vary in length ranging from greater than 25, 50, 100, 200, 300, 400, 500, 600 or 700 nucleotides and up to and including the entire coding sequence.

By “functional equivalent” as applied to a polynucleotide or a protein is intended a polynucleotide or a protein of sufficient length to modulate the level of CHD protein activity in a plant cell. A polynucleotide functional equivalent can be in sense or antisense orientation.

By “variants” is intended substantially similar sequences. Generally, nucleic acid sequence variants of the invention will have at least 60%, 65%, 70%, 75%, 80%, 90%, 95% or 98% sequence identity to the native nucleotide sequence, wherein the % sequence identity is based on the entire inventive sequence and is determined by GAP 10 analysis using default parameters. Generally, polypeptide sequence variants of the invention will have at least about 60%, 65%, 70%, 75%, 80%, 85%, 90%, 95% or 98% sequence identity to the native protein, wherein the % sequence identity is based on the entire sequence and is determined by GAP 10 analysis using default parameters. GAP uses the algorithm of Needleman and Wunsch (J. Mol. Biol. 48:443-453, 1970) to find the alignment of two complete sequences that maximizes the number of matches and minimizes the number of gaps.

As used herein a “responsive cell” refers to a cell that exhibits a positive response to the introduction of CHD polypeptide or CHD polynucleotide compared to a cell that has not been introduced with CHD polypeptide or CHD polynucleotide. The response can be to enhance tissue culture response, induce somatic embryogenesis, induce apomixis, increase transformation efficiency or increase recovery of regenerated plants.

As used herein a “recalcitrant plant cell” is a plant cell that exhibits unsatisfactory tissue culture response, transformation efficiency or recovery of regenerated plants compared to model systems. In maize such a model system is GS3. Elite maize inbreds are typically recalcitrant. In soybeans such model systems are Peking or Jack.

As used herein “Transformation” includes stable transformation and transient transformation unless indicated otherwise.

As used herein “Stable Transformation” refers to the transfer of a nucleic acid fragment into a genome of a host organism (this includes both nuclear and organelle genomes) resulting in genetically stable inheritance. In addition to traditional methods, stable transformation includes the alteration of gene expression by any means including chimerplasty or transposon insertion.

As used herein “Transient Transformation” refers to the transfer of a nucleic acid fragment or protein into the nucleus (or DNA-containing organelle) of a host organism resulting in gene expression without integration and stable inheritance.

As used herein, a “CHD-silencing” construct as an expression cassette whose transcribed mRNA or translated protein will diminish the functional expression of active CHD in the cell. Such silencing can be achieved through expression of an antisense construct targeted against the CHD structural gene, a vector in which the CHD structural gene or a portion of this sequence is used to make a silencing hairpin (or where silencing hairpin is conjoined to the CHD sequence in some fashion), or where a CHD-overexpression cassette is used to co-suppress endogenous CHD levels. Reducing activity of endogenous CHD protein can also be achieved through expression of a transgene encoding an antibody (including single chain antibodies) directed against a critical functional domain within the CHD molecule (for example, an antibody that was raised against the chromo-domain of CHD).

### Nucleic Acids

Expression of CHD genes and their localization within the cell modulate their chromatin-organizing function. Several CHD1-binding sites have been found in the nuclear matrix attachment region from mouse chromosomes, suggesting that this protein binds to chromosomes, at least during certain stages of the cell cycle. When cells enter mitosis, CHD1 has been shown in mouse cells to be released into the cytoplasm.

In an effort to elucidate the effect of gibberellic acid on Arabidopsis root development, a group of scientists in UC Berkely (Sung's lab) and Carnegie Institute of Washington (Sommerville's lab) discovered an Arabidopsis mutant called pickle (pkl). The primary root meristem of the pkl plant has embryonic characteristics. Root tissues from pickle plants can regenerate new embryos and plants without hormone induction (Ogas, et al., (1997) Science 277:91-94). This observation suggested that the pkl gene serves as a key repressor for plant embryogenesis. The gene was mapped to a position near 48.4 on chromosome 2. The sequence of AtPickle was then published (Ogas, et al., (1999) PNAS 96:13839-13844) and was found to be a CHD3 homologue. Interestingly, the Arabidopsis gymnos (gym) mutant was recently found to be allelic to pkl. GYM (PKL) acts as a suppressor to repress genes that promote meristematic activities (Eshed, et al., (1999) Cell 99:199-209).

Since the identification of the first CHD gene (MmCHD1, Delmas, et al., (1993) PNAS 90:2414-2418), a total of 13 highly conserved genes have so far been isolated. AtPKL and AtPKL-related genes are the only CHD genes isolated from plants.

CHD genes are required for appropriate inhibition of the transcription of important genes during development. Most likely, they are also required to be nonfunctional during embryogenesis and/or cell division. For those cells in which the key repressors are still on, overexpression of downstream, stimulatory genes may not be able to overcome the repression and consequently, no enhancement of transformation would be observed. Thus, manipulation of key repressor genes such that the repressor activity is transiently inhibited (antisense, cosuppression, antibody, etc.) may be an approach to establish an environment of embryogenesis and/or organogenesis. Working alone or together with LEC1, RepA or CycD, this approach may improve transformation.

In addition, modulating specific aspects of developmental pathways such as embryogenesis can be used to create high oil crops. Moreover, the family of CHD genes can be used to specifically shut down gene expression by engineering of specific DNA binding domains.

In many cases of apomixis maternal tissues such as the nucellus or inner integument “bud off” producing somatic embryos. These embryos then develop normally into seed. Since meiosis and fertilization are circumvented, the plants developing from such seed are genetically identical to the maternal plant. Suppression of expression of the CHD gene in the nucellus integument, or in the megaspore mother cell is expected to trigger embryo formation from maternal tissues.

Producing a seed identical to the parent has many advantages. For example high yielding hybrids could be used in seed production to multiply identical copies of high yielding hybrid seed. This would greatly reduce seed cost as well as increase the number of genotypes that are commercially available. Genes can be evaluated directly in commercial hybrids since the progeny would not segregate. This would save years of back crossing.

Apomixis would also provide a method of containment of transgenes when coupled with male sterility. The construction of male sterile autonomous agamospermy would prevent genetically engineered traits from hybridizing with weedy relatives.

Gene stacking would be relatively easy with apomixis. Hybrids could be successively re-transformed with various new traits and propagated via apomixis. The traits would not need to be linked since apomixis avoids the problems associated with segregation.

Apomixis can provide a reduction in gene silencing. Gene silencing is frequently seen following meiotic divisions. Since meiotic divisions never occur, it may be possible to eliminate or reduce the frequency of gene silencing. Apomixis can also be used to stabilize desirable phenotypes with complex traits such as hybrid vigor. Such traits could easily be maintained and multiplied indefinitely via apomixis.

Suppression of the CHD gene in transformed cells appears to initiate embryo development and stimulate development of pre-existing embryos. Reduced expression of the CHD gene should stimulate growth of transformed cells, but also insure that transformed somatic embryos develop in a normal, viable fashion (increasing the capacity of transformed somatic embryos to germinate vigorously).

Suppression of the CHD gene will stimulate growth in cells with the potential to initiate or maintain embryogenic growth. Cells in established meristems or meristem-derive cell lineages may be less prone to undergo the transition to embryos.

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a monocot or dicot. Typical examples of monocots are corn, sorghum, barley, wheat, millet, rice, or turf grass. Typical dicots include soybeans, sunflower, canola, alfalfa, potato, or cassava.

Functional fragments included in the invention can be obtained using primers that selectively hybridize under stringent conditions. Primers are generally at least 12 bases in length and can be as high as 200 bases, but will generally be from 15 to 75, preferably from 15 to 50 bases. Functional fragments can be identified using a variety of techniques such as restriction analysis, Southern analysis, primer extension analysis, and DNA sequence analysis.

The present invention includes a plurality of polynucleotides that encode for the identical amino acid sequence. The degeneracy of the genetic code allows for such “silent variations” which can be used, for example, to selectively hybridize and detect allelic variants of polynucleotides of the present invention. Additionally, the present invention includes isolated nucleic acids comprising allelic variants. The term “allele” as used herein refers to a related nucleic acid of the same gene.

Variants of nucleic acids included in the invention can be obtained, for example, by oligonucleotide-directed mutagenesis, linker-scanning mutagenesis, mutagenesis using the polymerase chain reaction, and the like. See, for example, Ausubel, pages 8.0.3-8.5.9. Also, see generally, McPherson (ed.), DIRECTED MUTAGENESIS: A Practical Approach, (IRL Press, 1991). Thus, the present invention also encompasses DNA molecules comprising nucleotide sequences that have substantial sequence similarity with the inventive sequences.

Variants included in the invention may contain individual substitutions, deletions or additions to the nucleic acid or polypeptide sequences which alters, adds or deletes a single amino acid or a small percentage of amino acids in the encoded sequence is a “conservatively modified variant” where the alteration results in the substitution of an amino acid with a chemically similar amino acid. When the nucleic acid is prepared or altered synthetically, advantage can be taken of known codon preferences of the intended host.

The present invention also includes “shufflents” produced by sequence shuffling of the inventive polynucleotides to obtain a desired characteristic. Sequence shuffling is described in PCT publication No. 96/19256. See also, Zhang, J H, et al., (1997) Proc. Natl. Acad. Sci. USA 94:4504-4509.

The present invention also includes the use of 5′ and/or 3′ UTR regions for modulation of translation of heterologous coding sequences. Positive sequence motifs include translational initiation consensus sequences (Kozak, (1987) Nucleic Acids Res. 15:8125) and the 7-methylguanosine cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284).

Further, the polypeptide-encoding segments of the polynucleotides of the present invention can be modified to alter codon usage. Altered codon usage can be employed to alter translational efficiency. Codon usage in the coding regions of the polynucleotides of the present invention can be analyzed statistically using commercially available software packages such as “Codon Preference” available from the University of Wisconsin Genetics Computer Group (see, Devereaux, et al., (1984) Nucleic Acids Res. 12:387-395) or MacVector 4.1 (Eastman Kodak Co., New Haven, Conn.).

For example, the inventive nucleic acids can be optimized for enhanced expression in plants of interest. See, for example, EPA0359472; WO91/16432; Perlak, et al., (1991) Proc. Natl. Acad. Sci. USA 88:3324-3328; and Murray, et al., (1989) Nucleic Acids Res. 17:477-498. In this manner, the polynucleotides can be synthesized utilizing plant-preferred codons. See, for example, Murray, et al., (1989) Nucleic Acids Res. 17:477-498, the disclosure of which is incorporated herein by reference.

The present invention provides subsequences comprising isolated nucleic acids containing at least 20 contiguous bases of the inventive sequences. For example the isolated nucleic acid includes those comprising at least 20, 30, 40, 50, 60, 70, 80, 90, 100, 200, 300, 400 or 500 contiguous nucleotides of the inventive sequences. Subsequences of the isolated nucleic acid can be used to modulate or detect gene expression by introducing into the subsequences compounds which bind, intercalate, cleave and/or crosslink to nucleic acids.

The nucleic acids of the invention may conveniently comprise a multi-cloning site comprising one or more endonuclease restriction sites inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention.

A polynucleotide of the present invention can be attached to a vector, adapter, promoter, transit peptide or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known and extensively described in the art. For a description of such nucleic acids see, for example, Stratagene Cloning Systems, Catalogs 1995, 1996, 1997 (La Jolla, Calif.); and, Amersham Life Sciences, Inc, Catalog '97 (Arlington Heights, Ill.).

The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library.

Exemplary total RNA and mRNA isolation protocols are described in Plant Molecular Biology: A Laboratory Manual, Clark, Ed., Springer-Verlag, Berlin (1997); and, Current Protocols in Molecular Biology, Ausubel, et al., Eds., Greene Publishing and Wiley-Interscience, New York (1995). Total RNA and mRNA isolation kits are commercially available from vendors such as Stratagene (La Jolla, Calif.), Clonetech (Palo Alto, Calif.), Pharmacia (Piscataway, N.J.), and 5′-3′ (Paoli, Pa.). See also, U.S. Pat. Nos. 5,614,391; and, 5,459,253.

Typical cDNA synthesis protocols are well known to the skilled artisan and are described in such standard references as: Plant Molecular Biology: A Laboratory Manual, Clark, Ed., Springer-Verlag, Berlin (1997); and, Current Protocols in Molecular Biology, Ausubel, et al., Eds., Greene Publishing and Wiley-Interscience, New York (1995). cDNA synthesis kits are available from a variety of commercial vendors such as Stratagene or Pharmacia.

An exemplary method of constructing a greater than 95% pure full-length cDNA library is described by Carninci, et al., (1996) Genomics, 37:327-336. Other methods for producing full-length libraries are known in the art. See, e.g., Edery, et al., (1995) Mol. Cell. Biol. 15(6):3363-3371; and PCT Application WO 96/34981.

It is often convenient to normalize a cDNA library to create a library in which each clone is more equally represented. A number of approaches to normalize cDNA libraries are known in the art. Construction of normalized libraries is described in Ko, (1990) Nucl. Acids. Res. 18(19):5705-5711; Patanjali, et al., (1991) Proc. Natl. Acad. U.S.A. 88:1943-1947; U.S. Pat. Nos. 5,482,685 and 5,637,685; and Soares, et al., (1994) Proc. Natl. Acad. Sci. USA 91:9228-9232.

Subtracted cDNA libraries are another means to increase the proportion of less abundant cDNA species. See, Foote et al. in, Plant Molecular Biology: A Laboratory Manual, Clark, Ed., Springer-Verlag, Berlin (1997); Kho and Zarbl, (1991) Technique 3(2):58-63; Sive and St. John, (1988) Nucl. Acids Res. 16(22):10937; Current Protocols in Molecular Biology, Ausubel, et al., Eds., Greene Publishing and Wiley-Interscience, New York (1995); and, Swaroop, et al., (1991) Nucl. Acids Res. 19(8):1954. cDNA subtraction kits are commercially available. See, e.g., PCR-Select (Clontech).

To construct genomic libraries, large segments of genomic DNA are generated by random fragmentation. Examples of appropriate molecular biological techniques and instructions are found in Sambrook, et al., (1989) Molecular Cloning: A Laboratory Manual, 2nd Ed., Cold Spring Harbor Laboratory, Vols. 1-3 (1989), Methods in Enzymology, Vol. 152: Guide to Molecular Cloning Techniques, Berger and Kimmel, Eds., San Diego: Academic Press, Inc. (1987), Current Protocols in Molecular Biology, Ausubel, et al., Eds., Greene Publishing and Wiley-Interscience, New York (1995); Plant Molecular Biology: A Laboratory Manual, Clark, Ed., Springer-Verlag, Berlin (1997). Kits for construction of genomic libraries are also commercially available.

The cDNA or genomic library can be screened using a probe based upon the sequence of a nucleic acid of the present invention such as those disclosed herein. Probes may be used to hybridize with genomic DNA or cDNA sequences to isolate homologous polynucleotides in the same or different plant species. Those of skill in the art will appreciate that various degrees of stringency of hybridization can be employed in the assay; and either the hybridization or the wash medium can be stringent. The degree of stringency can be controlled by temperature, ionic strength, pH and the presence of a partially denaturing solvent such as formamide.

Typically, stringent hybridization conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50° C. Exemplary moderate stringency conditions include hybridization in 40 to 45% formamide, 1 M NaCl, 1% SDS at 37° C., and a wash in 0.5× to 1×SSC at 55° C. Exemplary high stringency conditions include hybridization in 50% formamide, 1 M NaCl, 1% SDS at 37° C., and a wash in 0.1×SSC at 60° C. Typically the time of hybridization is from 4 to 16 hours.

An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., Eds., Greene Publishing and Wiley-Interscience, New York (1995). Often, cDNA libraries will be normalized to increase the representation of relatively rare cDNAs.

The nucleic acids of the invention can be amplified from nucleic acid samples using amplification techniques. For instance, polymerase chain reaction (PCR) technology can be used to amplify the sequences of polynucleotides of the present invention and related polynucleotides directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Examples of techniques useful for in vitro amplification methods are found in Berger, Sambrook, and Ausubel, as well as Mullis, et al., U.S. Pat. No. 4,683,202 (1987); and, PCR Protocols A Guide to Methods and Applications, Innis, et al., Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products. PCR-based screening methods have also been described. Wilfinger, et al., describe a PCR-based method in which the longest cDNA is identified in the first step so that incomplete clones can be eliminated from study. BioTechniques, 22(3):481-486 (1997).

In one aspect of the invention, nucleic acids can be amplified from a plant nucleic acid library. The nucleic acid library may be a cDNA library, a genomic library, or a library generally constructed from nuclear transcripts at any stage of intron processing. Libraries can be made from a variety of plant tissues. Good results have been obtained using mitotically active tissues such as shoot meristems, shoot meristem cultures, embryos, callus and suspension cultures, immature ears and tassels, and young seedlings. The cDNAs of the present invention were obtained from immature zygotic embryo and regenerating callus libraries.

Alternatively, the sequences of the invention can be used to isolate corresponding sequences in other organisms, particularly other plants, more particularly, other monocots. In this manner, methods such as PCR, hybridization, and the like can be used to identify such sequences having substantial sequence similarity to the sequences of the invention. See, for example, Sambrook, et al., (1989) Molecular Cloning: A Laboratory Manual (2d ed., Cold Spring Harbor Laboratory Press, Plainview, N.Y.). and Innis, et al., (1990), PCR Protocols: A Guide to Methods and Applications (Academic Press, New York). Coding sequences isolated based on their sequence identity to the entire inventive coding sequences set forth herein or to fragments thereof are encompassed by the present invention.

The isolated nucleic acids of the present invention can also be prepared by direct chemical synthesis by methods such as the phosphotriester method of Narang, et al., (1979) Meth. Enzymol. 68:90-99; the phosphodiester method of Brown, et al., (1979) Meth. Enzymol. 68:109-151; the diethylphosphoramidite method of Beaucage, et al., (1981) Tetra. Lett. 22:1859-1862; the solid phase phosphoramidite triester method described by Beaucage and Caruthers, (1981) Tetra. Letts. 22(20):1859-1862, e.g., using an automated synthesizer, e.g., as described in Needham-VanDevanter, et al., (1984) Nucleic Acids Res. 12:6159-6168; and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is limited to sequences of about 100 bases, longer sequences may be obtained by the ligation of shorter sequences.

The nucleic acids of the present invention include those amplified using the following primer pairs: SEQ ID NOS: 3 and 4; 7 and 8; 11 and 12; 15 and 16; 19 and 20; 23 and 24; 27 and 28; 31 and 32; 35 and 36; and 39 and 40.

### Expression Cassettes

In another embodiment expression cassettes comprising isolated nucleic acids of the present invention are provided. An expression cassette will typically comprise a polynucleotide of the present invention operably linked to transcriptional initiation regulatory sequences which will direct the transcription of the polynucleotide in the intended host cell, such as tissues of a transformed plant.

The construction of such expression cassettes which can be employed in conjunction with the present invention is well known to those of skill in the art in light of the present disclosure. See, e.g., Sambrook, et al., (1989) Molecular Cloning: A Laboratory Manual; Cold Spring Harbor, N.Y.;; Gelvin, et al., (1990) Plant Molecular Biology Manual; Plant Biotechnology: Commercial Prospects and Problems, eds. Prakash, et al., (1993) Oxford & IBH Publishing Co.; New Delhi, India; and Heslot, et al., (1992) Molecular Biology and Genetic Engineering of Yeasts; CRC Press, Inc., USA; each incorporated herein in its entirety by reference.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Constitutive, tissue-preferred or inducible promoters can be employed. Examples of constitutive promoters include the cauliflower mosaic virus (CaMV) 35S transcription initiation region, the 1′- or 2′-promoter derived from T-DNA of Agrobacterium tumefaciens, the actin promoter, the ubiquitin promoter, the histone H2B promoter (Nakayama, et al., (1992) FEBS Lett 30:167-170), the Smas promoter, the cinnamyl alcohol dehydrogenase promoter (U.S. Pat. No. 5,683,439), the Nos promoter, the pEmu promoter, the rubisco promoter, the GRP1-8 promoter, and other transcription initiation regions from various plant genes known in the art.

Examples of inducible promoters are the Adh1 promoter which is inducible by hypoxia or cold stress, the Hsp70 promoter which is inducible by heat stress, the PPDK promoter which is inducible by light, the In2 promoter which is safener induced, the ERE promoter which is estrogen induced and the Pepcarboxylase promoter which is light induced.

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter, Boronat, A., Martinez, M. C., Reina, M., Puigdomenech, P. and Palau, J.; Isolation and sequencing of a 28 kD glutelin-2 gene from maize: Common elements in the 5′ flanking regions among zein and glutelin genes; Plant Sci. 47:95-102 (1986) and Reina, M., Ponte, I., Guillen, P., Boronat, A. and Palau, J., Sequence analysis of a genomic clone encoding a Zc2 protein from Zea mays W64 A, Nucleic Acids Res. 18(21):6426 (1990). See the following site relating to the waxy promoter: Kloesgen, R. B., Gierl, A., Schwarz-Sommer, Z. S, and Saedler, H., Molecular analysis of the waxy locus of Zea mays, Mol. Gen. Genet. 203:237-244 (1986). The disclosures of each of these are incorporated herein by reference in their entirety.

The barley or maize Nucl promoter, the maize Cim 1 promoter or the maize LTP2 promoter can be used to preferentially express in the nucellus. See for example, U.S. Ser. No. 60/097,233 filed Aug. 20, 1998 the disclosure of which is incorporated herein by reference.

Either heterologous or non-heterologous (i.e., endogenous) promoters can be employed to direct expression of the nucleic acids of the present invention. These promoters can also be used, for example, in expression cassettes to drive expression of antisense nucleic acids to reduce, increase, or alter concentration and/or composition of the proteins of the present invention in a desired tissue.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

An intron sequence can be added to the 5′ untranslated region or the coding sequence of the partial coding sequence to increase the amount of the mature message that accumulates. See for example, Buchman and Berg, (1988) Mol. Cell. Biol. 8:4395-4405; Callis, et al., (1987) Genes Dev. 1:1183-1200. Use of maize introns Adh1-S intron 1, 2, and 6, the Bronze-1 intron are known in the art. See generally, The Maize Handbook, Chapter 116, Freeling and Walbot, Eds., Springer, N.Y. (1994).

The vector comprising the sequences from a polynucleotide of the present invention will typically comprise a marker gene which confers a selectable phenotype on plant cells. Usually, the selectable marker gene will encode antibiotic or herbicide resistance. Suitable genes include those coding for resistance to the antibiotics spectinomycin and streptomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance.

Suitable genes coding for resistance to herbicides include those which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), those which act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron.

While useful in conjunction with the above antibiotic and herbicide-resistance selective markers (i.e. use of the CHD gene can increase transformation frequencies when using chemical selection), use of the CHD gene confers a growth advantage to transformed cells without the need for inhibitory compounds to retard non-transformed growth. Thus, CHD transformants are recovered based solely on their differential growth advantage.

Typical vectors useful for expression of genes in higher plants are well known in the art and include vectors derived from the tumor-inducing (Ti) plasmid of Agrobacterium tumefaciens described by Rogers, et al., (1987) Meth. In Enzymol. 153:253-277. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA 86:8402-8406. Another useful vector herein is plasmid pBI101.2 that is available from Clontech Laboratories, Inc. (Palo Alto, Calif.).

A variety of plant viruses that can be employed as vectors are known in the art and include cauliflower mosaic virus (CaMV), geminivirus, brome mosaic virus, and tobacco mosaic virus.

A polynucleotide of the present invention can be expressed in either sense or anti-sense orientation as desired. In plant cells, it has been shown that antisense RNA inhibits gene expression by preventing the accumulation of mRNA which encodes the enzyme of interest, see, e.g., Sheehy, et al., (1988) Proc. Natl. Acad. Sci. USA 85:8805-8809; and Hiatt, et al., U.S. Pat. No. 4,801,340; U.S. Pat. No. 5,107,065; and U.S. Pat. No. 5,759,829.

Another method of suppression is sense suppression. Introduction of nucleic acid configured in the sense orientation has been shown to be an effective means by which to block the transcription of target genes. For an example of the use of this method to modulate expression of endogenous genes see, Napoli, et al., (1990) The Plant Cell 2:279-289 and U.S. Pat. No. 5,034,323. Recent work has shown suppression with the use of double stranded RNA. Such work is described in Tabara, et al., (1998) Science 282:5388:430-431; U.S. Pat. No. 6,506,559; and U.S. 2003/0056235 published Mar. 20, 2003. Hairpin approaches of gene suppression are disclosed in WO 98/53083 and WO 99/53050.

Catalytic RNA molecules or ribozymes can also be used to inhibit expression of plant genes. The inclusion of ribozyme sequences within antisense RNAs confers RNA-cleaving activity upon them, thereby increasing the activity of the constructs. The design and use of target RNA-specific ribozymes is described in Haseloff, et al., (1988) Nature 334:585-591.

A variety of cross-linking agents, alkylating agents and radical generating species as pendant groups on polynucleotides of the present invention can be used to bind, label, detect, and/or cleave nucleic acids. For example, Vlassov, V. V., et al., (1986) Nucleic Acids Res 14:4065-4076, describe covalent bonding of a single-stranded DNA fragment with alkylating derivatives of nucleotides complementary to target sequences. A report of similar work by the same group is that by Knorre, D. G., et al., (1985) Biochimie 67:785-789. Iverson and Dervan also showed sequence-specific cleavage of single-stranded DNA mediated by incorporation of a modified nucleotide which was capable of activating cleavage (J. Am. Chem. Soc. (1987) 109:1241-1243). Meyer, R B, et al., (1989) J. Am. Chem. Soc. 111:8517-8519, effect covalent crosslinking to a target nucleotide using an alkylating agent complementary to the single-stranded target nucleotide sequence. A photoactivated crosslinking to single-stranded oligonucleotides mediated by psoralen was disclosed by Lee, B L, et al., (1988) Biochemistry 27:3197-3203. Use of crosslinking in triple-helix forming probes was also disclosed by Home, et al., (1990) J. Am. Chem. Soc. 112:2435-2437. Use of N4, N4-ethanocytosine as an alkylating agent to crosslink to single-stranded oligonucleotides has also been described by Webb and Matteucci, (1986) J. Am. Chem. Soc. 108:2764-2765; Nucleic Acids Res (1986) 14:7661-7674; Feteritz, et al., (1991) J. Am. Chem. Soc. 113:4000. Various compounds to bind, detect, label, and/or cleave nucleic acids are known in the art. See, for example, U.S. Pat. Nos. 5,543,507; 5,672,593; 5,484,908; 5,256,648; and, 5,681,941.

### Proteins

CHD proteins are named for the three functional domains they contain. These include: a modifier of chromatin organization, a helicase/ATPase domain (similar to the chromatin-remodeling factor (SNF2) first found in yeast, named after a “sucrose non-fermenting” mutant, and a DNA-binding domain. CHD proteins are suggested to be involved in a range of basic processes including modification of chromatin structure, DNA repair, regulation of transcription, etc. In particular, CHD proteins inhibit transcription probably by binding to relatively long AT tracts in double-stranded DNA via minor-groove interactions. CHD proteins fall into two sub-families. CHD1 and CHD2 belong to the first sub-family while CHD3 and CHD4 belong to the second sub-family. A major difference between these two sub-families is that the CHD of the second sub-family has a zinc-finger domain in the N-terminal end which was thought to interact with histone deacetylases. Another feature is that the DNA-binding regions of the second sub-family members are more divergent than those of the first sub-family members.

Proteins of the present invention include proteins having the disclosed sequences as well proteins coded by the disclosed polynucleotides. In addition proteins of the present invention include proteins derived from the native protein by deletion (so-called truncation), addition or substitution of one or more amino acids at one or more sites in the native protein. Such variants may result from, for example, genetic polymorphism or from human manipulation. Methods for such manipulations are generally known in the art.

For example, amino acid sequence variants of the polypeptide can be prepared by mutations in the cloned DNA sequence encoding the native protein of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel, et al., (1987) Methods Enzymol. 154:367-382; Sambrook, et al., (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the protein of interest may be found in the model of Dayhoff, et al., (1978) Atlas of Protein Sequence and Structure (Natl. Biomed. Res. Found., Washington, D.C.), herein incorporated by reference. Conservative substitutions, such as exchanging one amino acid with another having similar properties, may be preferred.

In constructing variants of the proteins of interest, modifications to the nucleotide sequences encoding the variants will generally be made such that variants continue to possess the desired activity.

The isolated proteins of the present invention include a polypeptide comprising at least 30 contiguous amino acids encoded by any one of the nucleic acids of the present invention, or polypeptides that are conservatively modified variants thereof. The proteins of the present invention or variants thereof can comprise any number of contiguous amino acid residues from a polypeptide of the present invention, wherein that number is selected from the group of integers consisting of from 25 to the number of residues in a full-length polypeptide of the present invention. Optionally, this subsequence of contiguous amino acids is at least 25, 30, 40, 50, 60, 70, 80, 90, 100, 150, 200, 250, 300, 350, 400, 450 or 500 amino acids in length.

The present invention includes catalytically active polypeptides (i.e., enzymes). Catalytically active polypeptides will generally have a specific activity of at least about 20%, 30%, 40%, 50%, 60%, 70%, 80%, 90% or 95% that of the native (non-synthetic), endogenous polypeptide. Further, the substrate specificity (k_{cat}/Kₘ) is optionally substantially similar to the native (non-synthetic), endogenous polypeptide. Typically, the Kₘ will be at least about 30%, 40%, 50%, 60%, 70%, 80%, 90% or 95% that of the native (non-synthetic), endogenous polypeptide. Methods of assaying and quantifying measures of enzymatic activity and substrate specificity (k_{cat}/Kₘ), are well known to those of skill in the art.

The present invention includes modifications that can be made to an inventive protein. In particular, it may be desirable to diminish the activity of the gene. Other modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Using the nucleic acids of the present invention, one may express a protein of the present invention in recombinantly engineered cells such as bacteria, yeast, insect, mammalian, or plant cells. The cells produce the protein in a non-natural condition (e.g., in quantity, composition, location, and/or time), because they have been genetically altered through human intervention to do so.

Typically, an intermediate host cell will be used in the practice of this invention to increase the copy number of the cloning vector. With an increased copy number, the vector containing the gene of interest can be isolated in significant quantities for introduction into the desired plant cells.

Host cells that can be used in the practice of this invention include prokaryotes and eukaryotes. Prokaryotes include bacterial hosts such as Eschericia coli, Salmonella typhimurium, and Serratia marcescens. Eukaryotic hosts such as yeast or filamentous fungi may also be used in this invention. Since these hosts are also microorganisms, it will be essential to ensure that plant promoters which do not cause expression of the polypeptide in bacteria are used in the vector.

Commonly used prokaryotic control sequences include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Expression systems for expressing a protein of the present invention are available using Bacillus sp. and Salmonella (Palva, et al., (1983) Gene 22:229-235; Mosbach, et al., (1983) Nature 302:543-545).

Synthesis of heterologous proteins in yeast is well known. See, Sherman, F, et al., (1982) Methods in Yeast Genetics, Cold Spring Harbor Laboratory. Two widely utilized yeast for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysates. The monitoring of the purification process can be accomplished by using Western blot techniques or radioimmunoassay of other standard immunoassay techniques.

The proteins of the present invention can also be constructed using non-cellular synthetic methods. Solid phase synthesis of proteins of less than about 50 amino acids in length may be accomplished by attaching the C-terminal amino acid of the sequence to an insoluble support followed by sequential addition of the remaining amino acids in the sequence. Techniques for solid phase synthesis are described by Barany and Merrifield, Solid-Phase Peptide Synthesis, pp. 3-284 in The Peptides: Analysis, Synthesis, Biology. Vol. 2: Special Methods in Peptide Synthesis, Part A.; Merrifield, et al., (1963) J. Am. Chem. Soc. 85:2149-2156, and Stewart, et al., Solid Phase Peptide Synthesis, 2nd ed., Pierce Chem. Co., Rockford, III. (1984). Proteins of greater length may be synthesized by condensation of the amino and carboxy termini of shorter fragments. Methods of forming peptide bonds by activation of a carboxy terminal end (e.g., by the use of the coupling reagent N,N′-dicyclohexylcarbodiimide)) is known to those of skill.

The proteins of this invention, recombinant or synthetic, may be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503. Detection of the expressed protein is achieved by methods known in the art and include, for example, radioimmunoassays, Western blotting techniques or immunoprecipitation.

The present invention further provides a method for modulating (i.e., increasing or decreasing) the concentration or composition of the polypeptides of the present invention in a plant or part thereof. Modulation can be effected by increasing or decreasing the concentration and/or the composition (i.e., the ratio of the polypeptides of the present invention) in a plant.

The method comprises transforming a plant cell with an expression cassette comprising a polynucleotide of the present invention to obtain a transformed plant cell, growing the transformed plant cell under conditions allowing expression of the polynucleotide in the plant cell in an amount sufficient to modulate concentration and/or composition in the plant cell.

In some embodiments, the content and/or composition of polypeptides of the present invention in a plant may be modulated by altering, in vivo or in vitro, the promoter of a non-isolated gene of the present invention to up- or down-regulate gene expression. In some embodiments, the coding regions of native genes of the present invention can be altered via substitution, addition, insertion, or deletion to decrease activity of the encoded enzyme. See, e.g., Kmiec, U.S. Pat. No. 5,565,350; Zarling, et al., PCT/US93/03868. One method of down-regulation of the protein involves using PEST sequences that provide a target for degradation of the protein.

In some embodiments, an isolated nucleic acid (e.g., a vector) comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or composition of polypeptides of the present invention in the plant. Plant forming conditions are well known in the art.

In general, content of the polypeptide is increased or decreased by at least 5%, 10%, 20%, 30%, 40%, 50%, 60%, 70%, 80% or 90% relative to a native control plant, plant part, or cell lacking the aforementioned expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds which activate expression from these promoters are well known in the art. In preferred embodiments, the polypeptides of the present invention are modulated in monocots or dicots, preferably maize, soybeans, sunflower, sorghum, canola, wheat, alfalfa, rice, barley and millet.

Means of detecting the proteins of the present invention are not critical aspects of the present invention. In a preferred embodiment, the proteins are detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology, Vol. 37: Antibodies in Cell Biology, Asai, Ed., Academic Press, Inc. New York (1993); Basic and Clinical Immunology 7th Edition, Stites & Terr, Eds. (1991). Moreover, the immunoassays of the present invention can be performed in any of several configurations, e.g., those reviewed in Enzyme Immunoassay, Maggio, Ed., CRC Press, Boca Raton, Fla. (1980); Tijan, Practice and Theory of Enzyme Immunoassays, Laboratory Techniques in Biochemistry and Molecular Biology, Elsevier Science Publishers B. V., Amsterdam (1985); Harlow and Lane, supra; Immunoassay: A Practical Guide, Chan, Ed., Academic Press, Orlando, Fla. (1987); Principles and Practice of Immunoassays, Price and Newman Eds., Stockton Press, NY (1991); and Non-isotopic Immunoassays, Ngo, Ed., Plenum Press, NY (1988).

Typical methods include Western blot (immunoblot) analysis, analytic biochemical methods such as electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-1 inked immunosorbent assays (ELISAs), immunofluorescent assays, and the like.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904, which is incorporated herein by reference.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

The proteins of the present invention can be used for identifying compounds that bind to (e.g., substrates), and/or increase or decrease (i.e., modulate) the enzymatic activity of, catalytically active polypeptides of the present invention. The method comprises contacting a polypeptide of the present invention with a compound whose ability to bind to or modulate enzyme activity is to be determined. The polypeptide employed will have at least 20%, 30%, 40%, 50%, 60%, 70%, 80%, 90% or 95% of the specific activity of the native, full-length polypeptide of the present invention (e.g., enzyme). Methods of measuring enzyme kinetics are well known in the art. See, e.g., Segel, Biochemical Calculations, 2^{nd} ed., John Wiley and Sons, New York (1976).

Antibodies can be raised to a protein of the present invention, including individual, allelic, strain, or species variants, and fragments thereof, both in their naturally occurring (full-length) forms and in recombinant forms. Additionally, antibodies are raised to these proteins in either their native configurations or in non-native configurations. Anti-idiotypic antibodies can also be generated. Many methods of making antibodies are known to persons of skill.

In some instances, it is desirable to prepare monoclonal antibodies from various mammalian hosts, such as mice, rodents, primates, humans, etc. Description of techniques for preparing such monoclonal antibodies are found in, e.g., Basic and Clinical Immunology, 4th ed., Stites, et al., Eds., Lange Medical Publications, Los Altos, Calif., and references cited therein; Harlow and Lane, Supra; Goding, Monoclonal Antibodies: Principles and Practice, 2nd ed., Academic Press, New York, N.Y. (1986); and Kohler and Milstein, (1975) Nature 256:495-497.

Other suitable techniques involve selection of libraries of recombinant antibodies in phage or similar vectors (see, e.g., Huse, et al., (1989) Science 246:1275-1281; and Ward, et al., (1989) Nature 341:544-546; and Vaughan, et al., (1996) Nature Biotechnology 14:309-314). Alternatively, high avidity human monoclonal antibodies can be obtained from transgenic mice comprising fragments of the unrearranged human heavy and light chain Ig loci (i.e., minilocus transgenic mice). Fishwild, et al., (1996) Nature Biotech. 14:845-851. Also, recombinant immunoglobulins may be produced. See, Cabilly, U.S. Pat. No. 4,816,567; and Queen, et al., (1989) Proc. Natl. Acad. Sci. 86:10029-10033 (1989).

The antibodies of this invention can be used for affinity chromatography in isolating proteins of the present invention, for screening expression libraries for particular expression products such as normal or abnormal protein or for raising anti-idiotypic antibodies which are useful for detecting or diagnosing various pathological conditions related to the presence of the respective antigens.

Frequently, the proteins and antibodies of the present invention will be labeled by joining, either covalently or non-covalently, a substance which provides for a detectable signal. A wide variety of labels and conjugation techniques are known and are reported extensively in both the scientific and patent literature. Suitable labels include radionucleotides, enzymes, substrates, cofactors, inhibitors, fluorescent moieties, chemiluminescent moieties, magnetic particles, and the like.

### Transformation of Cells

The method of transformation is not critical to the present invention; various methods of transformation are currently available. As newer methods are available to transform crops or other host cells they may be directly applied. Accordingly, a wide variety of methods have been developed to insert a DNA sequence into the genome of a host cell to obtain the transcription and/or translation of the sequence to effect phenotypic changes in the organism. Thus, any method which provides for efficient transformation/transfection may be employed.

A DNA sequence coding for the desired polynucleotide of the present invention, for example a cDNA or a genomic sequence encoding a full length protein, can be used to construct an expression cassette which can be introduced into the desired plant. Isolated nucleic acid acids of the present invention can be introduced into plants according techniques known in the art. Generally, expression cassettes as described above and suitable for transformation of plant cells are prepared.

Techniques for transforming a wide variety of higher plant species are well known and described in the technical, scientific, and patent literature. See, for example, Weising, et al., (1988) Ann. Rev. Genet. 22:421-477. For example, the DNA construct may be introduced directly into the genomic DNA of the plant cell using techniques such as electroporation, PEG poration, particle bombardment, silicon fiber delivery, or microinjection of plant cell protoplasts or embryogenic callus. See, e.g., Tomes, et al., Direct DNA Transfer into Intact Plant Cells Via Microprojectile Bombardment. pp. 197-213 in Plant Cell, Tissue and Organ Culture, Fundamental Methods. eds. O. L. Gamborg and G. C. Phillips. Springer-Verlag Berlin Heidelberg New York, 1995. See also, Gordon-Kamm, et al., “Transformation of Zygote, Egg or Sperm Cells and Recovery of Transformed Plants from Isolated Embryos sacs”, U.S. Pat. No. 6,300,543. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616.

The introduction of DNA constructs using polyethylene glycol precipitation is described in Paszkowski, et al., (1984) Embo J. 3:2717-2722. Electroporation techniques are described in Fromm, et al., (1985) Proc. Natl. Acad. Sci. 82:5824. Ballistic transformation techniques are described in Klein, et al., (1987) Nature 327:70-73.

Agrobacterium tumefaciens-meditated transformation techniques are well described in the scientific literature. See, for example, Horsch, et al., (1984) Science 233:496-498, and Fraley, et al., (1983) Proc. Natl. Acad. Sci. 80:4803. For instance, Agrobacterium transformation of maize is described in U.S. Pat. No. 5,981,840. Agrobacterium transformation of soybean is described in U.S. Pat. No. 5,563,055.

Other methods of transformation include (1) Agrobacterium rhizogenes-mediated transformation (see, e.g., Lichtenstein and Fuller In: Genetic Engineering, Vol. 6, P W J Rigby, Ed., London, Academic Press, 1987; and Lichtenstein, C P, and Draper, J In: DNA Cloning, Vol. II, D. M. Glover, Ed., Oxford, IRI Press, 1985), Application PCT/US87/02512 (WO 88/02405 published Apr. 7, 1988) describes the use of A. rhizogenes strain A4 and its Ri plasmid along with A. tumefaciens vectors pARC8 or pARC16 (2) liposome-mediated DNA uptake (see, e.g., Freeman, et al., (1984) Plant Cell Physiol. 25:1353), (3) the vortexing method (see, e.g., Kindle, (1990) Proc. Natl. Acad. Sci. USA 87:1228).

DNA can also be introduced into plants by direct DNA transfer into pollen as described by Zhou, et al., (1983) Methods in Enzymology 101:433; D. Hess, (1987) Intern Rev. Cytol. 107:367; Luo, et al., (1988) Plant Mol. Biol. Reporter 6:165. Expression of polypeptide coding polynucleotides can be obtained by injection of the DNA into reproductive organs of a plant as described by Pena, et al., (1987) Nature 325:274. DNA can also be injected directly into the cells of immature embryos and the rehydration of desiccated embryos as described by Neuhaus, et al., (1987) Theor. Appl. Genet. 75:30; and Benbrook, et al., in Proceedings Bio Expo 1986, Butterworth, Stoneham, Mass., pp. 27-54 (1986).

Animal and lower eukaryotic (e.g., yeast) host cells are competent or rendered competent for transformation by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics, and micro-injection of the DNA directly into the cells. The transfected cells are cultured by means well known in the art. Kuchler, R J, Biochemical Methods in Cell Culture and Virology, Dowden, Hutchinson and Ross, Inc. (1977).

### Altering the Culture Medium to Suppress Somatic Embryogenesis in Non-Transformed Plant Cells and/or Tissues to Provide for a Positive Section Means of Transformed Plant Cells

Using the following methods for controlling somatic embryogenesis, it is possible to alter plant tissue culture media components to suppress somatic embryogenesis in a plant species of interest (often having multiple components that potentially could be adjusted to impart this effect). Such conditions would not impart a negative or toxic in vitro environment for wild-type tissue, but instead would simply not produce a somatic embryogenic growth form. Suppressing the expression of the CHD gene will stimulate somatic embryogenesis and growth in the transformed cells or tissue, providing a clear differential growth screen useful for identifying transformants.

Altering a wide variety of media components can modulate somatic embryogenesis (either stimulating or suppressing embryogenesis depending on the species and particular media component). Examples of media components which, when altered, can stimulate or suppress somatic embryogenesis include;
1) the basal medium itself (macronutrient, micronutrients and vitamins; see, T. A. Thorpe, 1981 for review, “Plant Tissue Culture: Methods and Applications in Agriculture”, Academic Press, NY),
2) plant phytohormones such as auxins (indole acetic acid, indole butyric acid, 2,4-dichlorophenoxyacetic acid, naphthaleneacetic acid, picloram, dicamba and other functional analogues), cytokinins (zeatin, kinetin, benzyl amino purine, 2-isopentyl adenine and functionally-related compounds) abscisic acid, adenine, and gibberellic acid,
3) and other compounds that exert “growth regulator” effects such as coconut water, casein hydrolysate, and proline, and
4) the type and concentration of gelling agent, pH and sucrose concentration.

Changes in the individual components listed above (or in some cases combinations of components) have been demonstrated in the literature to modulate in vitro somatic embryogenesis across a wide range of dicotyledonous and monocotyledonous species. For a compilation of examples, see, E. F. George, et al., (1987) Plant Tissue Culture Media, Vol. 1: Formulations and Uses. Exergetics, Ltd., Publ., Edington, England.

### Transgenic Plant Regeneration

Transformed plant cells which are derived by any of the above transformation techniques can be cultured to regenerate a whole plant which possesses the transformed genotype. Such regeneration techniques often rely on manipulation of certain phytohormones in a tissue culture growth medium, typically relying on a biocide and/or herbicide marker that has been introduced together with a polynucleotide of the present invention. For transformation and regeneration of maize see, Gordon-Kamm, et al., (1990) The Plant Cell 2:603-618.

Plants cells transformed with a plant expression vector can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans, et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants containing the foreign gene introduced by Agrobacterium can be achieved as described by Horsch, et al., (1985) Science, 227:1229-1231 and Fraley, et al., (1983) Proc. Natl. Acad. Sci. U.S.A. 80:4803. This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

Regeneration can also be obtained from plant callus, explants, organs, or parts thereof. Such regeneration techniques are described generally in Klee, et al., (1987) Ann. Rev. of Plant Phys. 38:467-486. The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). For maize cell culture and regeneration see generally, The Maize Handbook, Freeling and Walbot, Eds., Springer, N.Y. (1994); Corn and Corn Improvement, 3^{rd} edition, Sprague and Dudley Eds., American Society of Agronomy, Madison, Wis. (1988).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings, via production of apomictic seed, or by tissue culture techniques to produce multiple identical plants. Selection of desirable transgenics is made and new varieties are obtained and propagated vegetatively for commercial use. In seed propagated crops, mature transgenic plants can be self crossed to produce a homozygous inbred plant. The inbred plant produces seed containing the newly introduced heterologous nucleic acid. These seeds can be grown to produce plants that would produce the selected phenotype.

Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

Transgenic plants expressing a selectable marker can be screened for transmission of the nucleic acid of the present invention by, for example, standard immunoblot and DNA detection techniques. Transgenic lines are also typically evaluated on levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then be analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

A preferred embodiment is a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Back-crossing to a parental plant and out-crossing with a non-transgenic plant are also contemplated. Alternatively, propagation of heterozygous transgenic plants could be accomplished through apomixis.

The present invention provides a method of genotyping a plant comprising a polynucleotide of the present invention. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., Plant Molecular Biology: A Laboratory Manual, Chapter 7, Clark, Ed., Springer-Verlag, Berlin (1997). For molecular marker methods, see generally, The DNA Revolution by Andrew H. Paterson 1996 (Chapter 2) in: Genome Mapping in Plants (ed. Andrew H. Paterson) by Academic Press/R. G. Landis Company, Austin, Tex., pp. 7-21.

The particular method of genotyping in the present invention may employ any number of molecular marker analytic techniques such as, but not limited to, restriction fragment length polymorphisms (RFLPs). RFLPs are the product of allelic differences between DNA restriction fragments caused by nucleotide sequence variability. Thus, the present invention further provides a means to follow segregation of a gene or nucleic acid of the present invention as well as chromosomal sequences genetically linked to these genes or nucleic acids using such techniques as RFLP analysis.

Plants which can be used in the method of the invention include monocotyledonous and dicotyledonous plants. Preferred plants include maize, wheat, rice, barley, oats, sorghum, millet, rye, soybean, sunflower, alfalfa, canola, cotton, or turf grass.

Seeds derived from plants regenerated from transformed plant cells, plant parts or plant tissues, or progeny derived from the regenerated transformed plants, may be used directly as feed or food, or further processing may occur.

All publications cited in this application are herein incorporated by reference to the same extent as if each individual publication or patent application was specifically and individually indicated to be incorporated by reference.

The present invention will be further described by reference to the following detailed examples. It is understood, however, that there are many extensions, variations, and modifications on the basic theme of the present invention beyond that shown in the examples and description, which are within the spirit and scope of the present invention.

### EXAMPLES

### Example 1

### Library Construction Used for the Maize CHD EST's

### A. Total RNA Isolation

Total RNA was isolated from maize embryo and regenerating callus tissues with TRIzol Reagent (Life Technology Inc. Gaithersburg, Md.) using a modification of the guanidine isothiocyanate/acid-phenol procedure described by Chomczynski and Sacchi (Chomczynski, P and Sacchi, N, (1987) Anal. Biochem 162:156). In brief, plant tissue samples were pulverized in liquid nitrogen before the addition of the TRIzol Reagent, and then were further homogenized with a mortar and pestle. Addition of chloroform followed by centrifugation was conducted for separation of an aqueous phase and an organic phase. The total RNA was recovered by precipitation with isopropyl alcohol from the aqueous phase.

### B. Poly(A)+ RNA Isolation

The selection of poly(A)+ RNA from total RNA was performed using PolyATact system (Promega Corporation. Madison, Wis.). In brief, biotinylated oligo(dT) primers were used to hybridize to the 3′ poly(A) tails on mRNA. The hybrids were captured using streptavidin coupled to paramagnetic particles and a magnetic separation stand. The mRNA was washed at high stringent condition and eluted by RNase-free deionized water.

### C. cDNA Library Construction

cDNA synthesis was performed and unidirectional cDNA libraries were constructed using the SuperScript Plasmid System (Life Technology Inc. Gaithersburg, Md.). The first stand of cDNA was synthesized by priming an oligo(dT) primer containing a Not I site. The reaction was catalyzed by SuperScript Reverse Transcriptase II at 45° C. The second strand of cDNA was labeled with alpha-³²P-dCTP and a portion of the reaction was analyzed by agarose gel electrophoresis to determine cDNA sizes. cDNA molecules smaller than 500 base pairs and unligated adapters were removed by Sephacryl-S400 chromatography. The selected cDNA molecules were ligated into pSPORT1 vector in between of Not I and Sal I sites.

### D. Genomic library Construction into BAC (Bacterial Artificial Chromosome) Vectors.

BAC library were constructed according Texas A&M BAC center protocol. High molecular weight DNA isolated from line Mo17 embedded in LMP agarose microbeads were partially digested by HindIII. After partial digestion, the DNA was size-selected pulsed-field gel electrophoresis to remove the smaller DNA fragments that can compete more effectively than the larger DNA fragments for vector ends. The size-selected DNA fragments were ligated into pBeloBAC11 in HindIII site.

### Example 2

### Sequencing and cDNA Subtraction Procedures Used for Maize CHD EST's

### A. Sequencing Template Preparation

Individual colonies were picked and DNA was prepared either by PCR with M13 forward primers and M13 reverse primers, or by plasmid isolation. All the cDNA clones were sequenced using M13 reverse primers.

### B. Q-bot Subtraction Procedure

cDNA libraries subjected to the subtraction procedure were plated out on 22×22 cm² agar plate at density of about 3,000 colonies per plate. The plates were incubated in a 37° C. incubator for 12-24 hours. Colonies were picked into 384-well plates by a robot colony picker, Q-bot (GENETIX Limited). These plates were incubated overnight at 37° C.

Once sufficient colonies were picked, they were pinned onto 22×22 cm² nylon membranes using Q-bot. Each membrane contained 9,216 colonies or 36,864 colonies. These membranes were placed onto agar plate with appropriate antibiotic. The plates were incubated at 37° C. for overnight.

After colonies were recovered on the second day, these filters were placed on filter paper prewetted with denaturing solution for four minutes, then were incubated on top of a boiling water bath for additional four minutes. The filters were then placed on filter paper prewetted with neutralizing solution for four minutes. After excess solution was removed by placing the filters on dry filter papers for one minute, the colony side of the filters were place into Proteinase K solution, incubated at 37° C. for 40-50 minutes. The filters were placed on dry filter papers to dry overnight. DNA was then cross-linked to nylon membrane by UV light treatment. Colony hybridization was conducted as described by Sambrook, J., Fritsch, E F and Maniatis, T, (in Molecular Cloning: A laboratory Manual, 2^{nd} Edition). The following probes were used in colony hybridization:
1. First strand cDNA from the same tissue from which the library was made to remove the most redundant clones.
2. 48-192 most redundant cDNA clones from the same library based on previous sequencing data.
3. 192 most redundant cDNA clones in the entire corn sequence database.
4. A Sal-A20 oligo nucleotide: TCG ACC CAC GCG TCC GAA AAA AAA AAA AAA AAA AAA, removes clones containing a poly A tail but no cDNA.
5. cDNA clones derived from rRNA.

The image of the autoradiography was scanned into computer and the signal intensity and cold colony addresses of each colony was analyzed. Re-arraying of cold-colonies from 384 well plates to 96 well plates was conducted using Q-bot.

### Example 3

### Identification of Maize CHD EST's from a Computer Homology Search

Gene identities were determined by conducting BLAST (Basic Local Alignment Search Tool; Altschul, S F, et al., (1993) J. Mol. Biol. 215:403-410; see also, www.ncbi.nlm.nih.gov/BLAST/) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences were analyzed for similarity to all publicly available DNA sequences contained in the “nr” database using the BLASTN algorithm. The DNA sequences were translated in all reading frames and compared for similarity to all publicly available protein sequences contained in the “nr” database using the BLASTX algorithm (Gish, W and States, D J, (1993) Nature Genetics 3:266-272) provided by the NCBI. In some cases, the sequencing data from two or more clones containing overlapping segments of DNA were used to construct contiguous DNA sequences.

### Example 4

### Transformation and Regeneration of Maize Callus

Expression vectors most useful for modulating CHD expression are those that down-regulate CHD levels or activity (abbreviated hereafter as CHD-DR constructs). A CHD-DR construct is an expression cassette in which the transcribed RNA results in decreased levels of CHD protein in the cell. Examples would include expressing antisense, expressing an inverted-repeat sequence (which will form a hairpin) constructed from a portion of the CHD sequence, expressing the CHD sequence fused to another such “hairpin” forming sequence, or expressing CHD in a manner that will favor co-suppression of endogenous CHD.

Transformation of a CHD-DR construct (whether antisense, hairpin, or co-suppression-based) along with a marker-expression cassette (for example, UBI::moPAT-GPFm::pinII) into genotype Hi-II follows a well-established bombardment transformation protocol used for introducing DNA into the scutellum of immature maize embryos (Songstad, D D, et al., (199) In Vitro Cell Dev. Biol. Plant 32:179-183). It is noted that any suitable method of transformation can be used, such as Agrobacterium-mediated transformation and many other methods. To prepare suitable target tissue for transformation, ears are surface sterilized in 50% Chlorox bleach plus 0.5% Micro detergent for 20 minutes, and rinsed two times with sterile water. The immature embryos (approximately 1-1.5 mm in length) are excised and placed embryo axis side down (scutellum side up), 25 embryos per plate. These are cultured onto medium containing N6 salts, Erikkson's vitamins, 0.69 g/l proline, 2 mg/l 2,4-D and 3% sucrose. After 4-5 days of incubation in the dark at 28° C., embryos are removed from the first medium and cultured onto similar medium containing 12% sucrose. Embryos are allowed to acclimate to this medium for 3 h prior to transformation. The scutellar surface of the immature embryos is targeted using particle bombardment. Embryos are transformed using the PDS-1000 Helium Gun from Bio-Rad at one shot per sample using 650 PSI rupture disks. DNA delivered per shot averages approximately 0.1667 μg. Following bombardment, all embryos are maintained on standard maize culture medium (N6 salts, Erikkson's vitamins, 0.69 g/l proline, 2 mg/l 2,4-D, 3% sucrose) for 2-3 days and then transferred to N6-based medium containing 3 mg/L Bialaphos®. Plates are maintained at 28° C. in the dark and are observed for colony recovery with transfers to fresh medium every two to three weeks. After approximately 10 weeks of selection, selection-resistant GFP positive callus clones are sampled for PCR and activity of the polynucleotide of interest. Positive lines are transferred to 288J medium, an MS-based medium with lower sucrose and hormone levels, to initiate plant regeneration. Following somatic embryo maturation (2-4 weeks), well-developed somatic embryos are transferred to medium for germination and transferred to the lighted culture room. Approximately 7-10 days later, developing plantlets are transferred to medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to Classic™ 600 pots (1.6 gallon) and grown to maturity. Plants are monitored for expression of the polynucleotide of interest. Recovered colonies and plants are scored based on GFP visual expression, leaf painting sensitivity to a 1% application of Ignite® herbicide, and molecular characterization via PCR and Southern analysis.

Transformation of a CHD-DR cassette along with UBI::moPAT˜moGFP::pinII into a maize genotype such as Hi-II (or inbreds such as Pioneer Hi-Bred International, Inc. proprietary inbreds N46 and P38) is also done using the Agrobacterium mediated DNA delivery method, as described by U.S. Pat. No. 5,981,840 with the following modifications. Again, it is noted that any suitable method of transformation can be used, such as particle-mediated transformation, as well as many other methods. Agrobacteria are grown to log phase in liquid minimal-A medium containing 100 μM spectinomycin. Embryos are immersed in a log phase suspension of Agrobacteria adjusted to obtain an effective concentration of 5×10⁸ cfu/ml. Embryos are infected for 5 minutes and then co-cultured on culture medium containing acetosyringone for 7 days at 20° C. in the dark. After 7 days, the embryos are transferred to standard culture medium (MS salts with N6 macronutrients, 1 mg/L 2,4-D, 1 mg/L Dicamba, 20 g/L sucrose, 0.6 g/L glucose, 1 mg/L silver nitrate, and 100 mg/L carbenicillin) with 3 mg/L Bialaphos® as the selective agent. Plates are maintained at 28° C. in the dark and are observed for colony recovery with transfers to fresh medium every two to three weeks. Positive lines are transferred to an MS-based medium with lower sucrose and hormone levels, to initiate plant regeneration. Following somatic embryo maturation (2-4 weeks), well-developed somatic embryos are transferred to medium for germination and transferred to the lighted culture room. Approximately 7-10 days later, developed plantlets are transferred to medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to Classic™ 600 pots (1.6 gallon) and grown to maturity. Recovered colonies and plants are scored based on GFP visual expression, leaf painting sensitivity to a 1% application of Ignite® herbicide, and molecular characterization via PCR and Southern analysis.

### A. Introducing CHD-DR to Improve Transformation Frequency Using Agrobacterium or Particle Bombardment.

Plasmids described in Example 4 are used to transform Hi-II immature embryos using particle delivery or the Agrobacterium. Bialaphos resistant GFP+ colonies are counted using a GFP microscope and transformation frequencies are determined (percentage of initial target embryos from which at least one GFP-expressing, bialaphos-resistant multicellular transformed event grows). In both particle gun experiments and Agrobacterium experiments, transformation frequencies are expected to increase with CHD treatment.

### B. Down-Regulation of CHD to Improve the Embryogenic Phenotype and Regeneration Capacity of Inbreds.

Immature embryos from the inbred P38 are isolated, cultured and transformed as described above, with the following changes. Embryos are initially cultured on 601H medium (a MS based medium with 0.1 mg/l zeatin, 2 mg/l 2,4-D, MS and SH vitamins, proline, silver nitrate, extra potassium nitrate, casein hydrolysate, gelrite, 10 g/l glucose and 20 g/l sucrose). Prior to bombardment embryos are moved to a high osmoticum medium (modified Duncan's with 2 mg/l 2,4-D and 12% sucrose). Post bombardment, embryos are moved to 601H medium with 3 mg/l bialaphos for two weeks. Embryos are then moved to 601H medium without proline and casein hydrolysate with 3 mg/l bialaphos and transferred every two weeks. Transformation frequency is determined by counting the numbers of bialaphos-resistant GFP-positive colonies. Colonies are also scored on whether they have an embryogenic (regenerable) or non-embryogenic phenotype. Compared to the control treatment (UBI::moPAT˜moGFP::pinII alone), treatments including the marker cassette (UBI::moPAT˜moGFP::pinII)+CHD-DR is expected to result in consistently higher transformation frequencies, the transformants having a more embryogenic callus phenotype and the frequency of successful regeneration from transformed callus should be substantially improved.

### Example 5

### Transient Suppression of the CHD Polynucleotide Product to Induce Somatic Embryogenesis

It may be desirable to “kick start” somatic embryogenesis by transiently expressing a CHD-DR polynucleotide product. This can be done by delivering CHD-DR 5′ capped polyadenylated RNA or expression cassettes containing CHD-DR DNA. These molecules can be delivered using a biolistics particle gun. For example 5′ capped polyadenylated CHD-DR RNA can easily be made in vitro using Ambion's mMessage mMachine kit. Following the procedure outline above RNA is co-delivered along with DNA containing an agronomically useful expression cassette. The cells receiving the RNA will form somatic embryos and a large portion of these will have integrated the agronomic gene. Plants regenerated from these embryos can then be screened for the presence of the agronomic gene.

### Example 6

### Use of the Maize CHD to Induce Apomixis

Maize expression cassettes down-regulating CHD expression in the inner integument or nucellus can easily be constructed. An expression cassette directing expression of the CHD-DR polynucleotide to the nucellus is made using the barley Nucl promoter. Embryos are co-bombarded with the selectable marker PAT fused to the GFP gene (UBI::moPAT˜moGFP) along with the nucellus specific CHD-DR expression cassette described above. Both inbred (P38) and GS3 transformants are obtained and regenerated as described in examples 4.

It is expected that the regenerated plants will then be capable of producing de novo embryos from CHD-DR expressing nucellar cells. This is complemented by pollinating the ears to promote normal central cell fertilization and endosperm development. In another variation of this scheme, nuc1:CHD-DR transformations could be done using a FIE-null genetic background which would promote both de novo embryo development and endosperm development without fertilization (see, Ohad, et al., (1999) The Plant Cell 11:407-415; also pending U.S. application Ser. No. 60/151,575 filed Aug. 31, 1999). Upon microscopic examination of the developing embryos it will be apparent that apomixis has occurred by the presence of embryos budding off the nucellus. In yet another variation of this scheme the CHD-DR polynucleotide could be delivered as described above into a homozygous zygotic-embryo-lethal genotype. Only the adventive embryos produced from somatic nucellus tissue would develop in the seed.

### Example 7

### Expression of Chimeric Genes in Microbial Cells

The cDNAs encoding the instant transcription factors can be inserted into the T7 E. coli expression vector pBT430. This vector is a derivative of pET-3a (Rosenberg, et al., (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% NuSieve GTG™ low melting agarose gel (FMC). Buffer and agarose contain 10 μg/ml ethidium bromide for visualization of the DNA fragment. The fragment can then be purified from the agarose gel by digestion with GELase™ (Epicentre Technologies) according to the manufacturer's instructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the polynucleotide encoding the transcription factor are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier, et al., (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 hours at 25° C. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

### Example 8

### Evaluating Compounds for their Ability to Inhibit the Activity of Plant Transcription Factors

The transcription factors described herein may be produced using any number of methods known to those skilled in the art. Such methods include, but are not limited to, expression in bacteria as described in Example 7, or expression in eukaryotic cell culture, in planta, and using viral expression systems in suitably infected organisms or cell lines. The instant transcription factors may be expressed either as mature forms of the proteins as observed in vivo or as fusion proteins by covalent attachment to a variety of enzymes, proteins or affinity tags. Common fusion protein partners include glutathione S-transferase (“GST”), thioredoxin (“Trx”), maltose binding protein, and C- and/or N-terminal hexahistidine polypeptide (“(His)₆”). The fusion proteins may be engineered with a protease recognition site at the fusion point so that fusion partners can be separated by protease digestion to yield intact mature enzyme. Examples of such proteases include thrombin, enterokinase and factor Xa. However, any protease can be used which specifically cleaves the peptide connecting the fusion protein and the enzyme.

Purification of the instant transcription factors, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. When the transcription factors are expressed as fusion proteins, the purification protocol may include the use of an affinity resin which is specific for the fusion protein tag attached to the expressed enzyme or an affinity resin containing ligands which are specific for the enzyme. For example, a transcription factor may be expressed as a fusion protein coupled to the C-terminus of thioredoxin. In addition, a (His)₆ peptide may be engineered into the N-terminus of the fused thioredoxin moiety to afford additional opportunities for affinity purification. Other suitable affinity resins could be synthesized by linking the appropriate ligands to any suitable resin such as Sepharose-4B. In an alternate embodiment, a thioredoxin fusion protein may be eluted using dithiothreitol; however, elution may be accomplished using other reagents which interact to displace the thioredoxin from the resin. These reagents include β-mercaptoethanol or other reduced thiol. The eluted fusion protein may be subjected to further purification by traditional means as stated above, if desired. Proteolytic cleavage of the thioredoxin fusion protein and the enzyme may be accomplished after the fusion protein is purified or while the protein is still bound to the ThioBond™ affinity resin or other resin.

Crude, partially purified or purified enzyme, either alone or as a fusion protein, may be utilized in assays for the evaluation of compounds for their ability to inhibit enzymatic activation of the transcription factors disclosed herein. Assays may be conducted under well-known experimental conditions that permit optimal enzymatic activity.

### Example 9

### CHD Down-Regulation to Increase Growth Rates, which could be Used as a Screening Criterion for Positive Selection of Transformants

Using two promoters of increasing strength to drive expression of CHD-DR cassettes in maize, it appears that CHD-DR stimulates callus growth over control treatments and the stronger promoter driving CHD-DR results in faster growth than with the low-level promoter. For example, an experiment is performed to compare the In2 and nos promoters. As noted above, based on our experience with these two promoters driving other genes, the In2 promoter (in the absence of an inducer other than auxin from the medium) would drive expression at very low levels. The nos promoter has been shown to drive moderately-low levels of transgene expression (approximately 10- to 30-fold lower than the maize ubiquitin promoter, but still stronger than In2 under the culture conditions used in this experiment). One control treatment is used in this experiment, the UBI:PAT˜GFPmo:pinII construct by itself (with no CHD-DR). Hi-II immature embryos are bombarded as previously described, and transgenic, growing events are scored at 3 and 6 weeks. The control treatment results in a typical transformation frequency, for example of 0.8%. The In2 and nos-driven CHD down-regulator treatments are expected to result in progressively higher transformation frequencies, for example 25 and 40%, respectively.

Within these treatments there is also expected an increase in the overall frequency of large, rapidly growing calli, relative to the control treatment. For this data, the fresh weight of transformed calli is recorded 2 months after bombardment. Assuming that all the transgenic events started as single transformed cells within a few days after bombardment, these weights represent the relative growth rate of these transformants during this period (all tissue is sub-cultured and weighed for each transformant; mean weights and standard deviations are calculated for each treatment). For the control treatment, the mean transformant weight after two months is expected to be 37+/−15 mg (n=6). For the In2:CHD-down-regulator and nos:CHD-down-regulator treatments, the mean transformant weights are expected to be 126+/−106 and 441+/−430 mg, respectively. If the control treatment is set at a relative growth value of 1.0, this means that transformants in the In2: CHD-down-regulator and nos: CHD-down-regulator treatments are expected to grow 3.4 and 12-fold faster than the control. Increasing CHD down regulation should result in a concomitant increase in callus growth rate.

### Example 10

### The Use of CHD Polynucleotide as a Positive Selection System for Wheat Transformation and for Improving the Regeneration Capacity of Wheat Tissues

### Method

### Plant Material

Seeds of wheat Hybrinova lines NH535 and BO 014 are sown into soil in plug trays for vernalisation at 6° C. for eight weeks. Vernalized seedlings are transferred in 8″ pots and grown in a controlled environment room. The growth conditions used are; 1) soil composition: 75% L&P fine-grade peat, 12% screened sterilized loam, 10% 6 mm screened, lime-free grit, 3% medium grade vermiculite, 3.5 kg Osmocote per m³ soil (slow-release fertilizer, 15-11-13 NPK plus micronutrients), 0.5 kg PG mix per m³ (14-16-18 NPK granular fertilizer plus micronutrients, 2) 16 h photoperiod (400 W sodium lamps providing irradiance of ca. 750 μE s⁻¹ m⁻²), 18 to 20° C. day and 14 to 16° C. night temperature, 50 to 70% relative air humidity and 3) pest control: sulfur spray every 4 to 6 weeks and biological control of thrips using Amblyseius caliginosus (Novartis BCM Ltd, UK).

### Isolation of Explants and Culture Initiation

Two sources of primary explants are used; scutellar and inflorescence tissues. For scutella, early-medium milk stage grains containing immature translucent embryos are harvested and surface-sterilized in 70% ethanol for 5 min. and 0.5% hypochlorite solution for 15-30 min. For inflorescences, tillers containing 0.5-1.0 cm inflorescences are harvested by cutting below the inflorescence-bearing node (the second node of a tiller). The tillers are trimmed to approximately 8-10 cm length and surface-sterilized as above with the upper end sealed with Nescofilm (Bando Chemical Ind. Ltd, Japan).

Under aseptic conditions, embryos of approximately 0.5-1.0 mm length are isolated and the embryo axis removed. Inflorescences are dissected from the tillers and cut into approximately 1 mm pieces. Thirty scutella or 1 mm inflorescence explants are placed in the center (18 mm target circle) of a 90 mm Petri dish containing MD0.5 or L7D2 culture medium. Embryos are placed with the embryo-axis side in contact with the medium exposing the scutellum to bombardment whereas inflorescence pieces are placed randomly. Cultures are incubated at ±25° C. in darkness for approximately 24 h before bombardment. After bombardment, explants from each bombarded plate are spread across three plates for callus induction.

### Culture Media

The standard callus induction medium for scutellar tissues (MD0.5) consists of solidified (0.5% Agargel, Sigma A3301) modified MS medium supplemented with 9% sucrose, 10 mg I⁻¹ AgNO₃ and 0.5 mg I⁻¹ 2,4-D (Rasco-Gaunt, et al., 1999). Inflorescence tissues are cultured on L7D2 which consists of solidified (0.5% Agargel) L3 medium supplemented with 9% maltose and 2 mg I⁻¹ 2,4-D (Rasco-Gaunt and Barcelo, 1999). The basal shoot induction medium, RZ contains L salts, vitamins and inositol, 3% w/v maltose, 0.1 mg I⁻¹ 2,4-D and 5 mg I⁻¹ zeatin (Rasco-Gaunt and Barcelo, 1999). Regenerated plantlets are maintained in RO medium with the same composition as RZ, but without 2,4-D and zeatin.

### DNA Precipitation Procedure and Particle Bombardment

Submicron gold particles (0.6 μm Micron Gold, Bio-Rad) are coated with a plasmid containing a CHD-DR construct following the protocol modified from the original Bio-Rad procedure (Barcelo and Lazzeri, 1995). The standard precipitation mixture consists of 1 mg of gold particles in 50 μl SDW, 50 μl of 2.5 M calcium chloride, 20 μl of 100 mM spermidine free base and 5 μl DNA (concentration 1 μg μl⁻¹). After combining the components, the mixture is vortexed and the supernatant discarded. The particles are then washed with 150 μl absolute ethanol and finally resuspended in 85 μl absolute ethanol. The DNA/gold ethanol solution is kept on ice to minimize ethanol evaporation. For each bombardment, 5 μl of DNA/gold ethanol solution (ca. 60 μg gold) is loaded onto the macrocarrier.

Particle bombardments are carried out using DuPont PDS 1000/He gun with a target distance of 5.5 cm from the stopping plate at 650 psi acceleration pressure and 28 in. Hg chamber vacuum pressure.

### Regeneration of Transformants

For callus induction, bombarded explants are distributed over the surface of the medium in the original dish and two other dishes and cultured at 25±1° C. in darkness for three weeks. Development of somatic embryos from each callus are periodically recorded. For shoot induction, calluses are transferred to RZ medium and cultured under 12 h light (250 μE S⁻¹ m⁻², from cool white fluorescent tubes) at 25±1° C. for three weeks for two rounds. All plants regenerating from the same callus are noted. Plants growing more vigorously than the control cultures are potted in soil after 6-9 weeks in R0 medium. The plantlets are acclimatized in a propagator for 1-2 weeks. Thereafter, the plants are grown to maturity under growth conditions described above.

### DNA Isolation from Callus and Leaf Tissues

Genomic DNA as extracted from calluses or leaves using a modification of the CTAB (cetyltrimethylammonium bromide, Sigma H5882) method described by Stacey and Isaac cite (1994). Approximately 100-200 mg of frozen tissues is ground into powder in liquid nitrogen and homogenized in 1 ml of CTAB extraction buffer (2% CTAB, 0.02 M EDTA, 0.1 M Tris-Cl pH 8, 1.4 M NaCl, 25 mM DTT) for 30 min at 65° C. Homogenized samples are allowed to cool at room temperature for 15 min before a single protein extraction with approximately 1 ml 24:1 v/v chloroform:octanol is done. Samples are centrifuged for 7 min at 13,000 rpm and the upper layer of supernatant collected using wide-mouthed pipette tips. DNA is precipitated from the supernatant by incubation in 95% ethanol on ice for 1 h. DNA threads are spooled onto a glass hook, washed in 75% ethanol containing 0.2 M sodium acetate for 10 min, air-dried for 5 min and resuspended in TE buffer. Five μl RNAse A is added to the samples and incubated at 37° C. for 1 h.

For quantification of genomic DNA, gel electrophoresis is performed using an 0.8% agarose gel in 1×TBE buffer. One microliter of the samples are fractionated alongside 200, 400, 600 and 800 ng μl⁻¹λ uncut DNA markers.

### Polymerase Chain Reaction (PCR) Analysis

The presence of the maize CHD-DR polynucleotide is analyzed by PCR using 100-200 ng template DNA in a 30 ml PCR reaction mixture containing 1× concentration enzyme buffer (10 mM Tris-HCl pH 8.8, 1.5 mM magnesium chloride, 50 mM potassium chloride, 0.1% Triton X-100), 200 μM dNTPs, 0.3 μM primers and 0.022 U TaqDNA polymerase (Boehringer Mannheim). Thermocycling conditions are as follows (30 cycles): denaturation at 95° C. for 30 s, annealing at 55° C. for 1 min and extension at 72° C. for 1 min.

After particle-mediated delivery of either a UBI::PAT˜GFPmo::pinII construct alone (control treatment), or the UBI::PAT˜GFPmo::pinII+CHD-DR, in the treatments containing the CDH-DR construct there should be a higher frequency of embryogenic transformants recovered and the regeneration capacity of these transformants should be substantially improved over the control treatment. In addition, in the CDH-DR treatment the frequency of escape colonies should be reduced.

### Example 11

### Expression of Chimeric Genes in Dicot Cells

The CHD-DR polynucleotide can also be used to improve the transformation of soybean. To demonstrate this the construct consisting of the In2 promoter and CHD-DR sequence are introduced into embryogenic suspension cultures of soybean by particle bombardment using essentially the methods described in Parrott, W. A., L. M. Hoffman, D. F. Hildebrand, E. G. Williams, and G. B. Collins, (1989) Recovery of primary transformants of soybean, Plant Cell Rep. 7:615-617. This method with modifications is described below.

Seed is removed from pods when the cotyledons are between 3 and 5 mm in length. The seeds are sterilized in a Chlorox solution (0.5%) for 15 minutes after which time the seeds are rinsed with sterile distilled water. The immature cotyledons are excised by first excising the portion of the seed that contains the embryo axis. The cotyledons are then removed from the seed coat by gently pushing the distal end of the seed with the blunt end of the scalpel blade. The cotyledons are then placed (flat side up) SB1 initiation medium (MS salts, B5 vitamins, 20 mg/L 2,4-D, 31.5 g/l sucrose, 8 g/L TC Agar, pH 5.8). The Petri plates are incubated in the light (16 hr day; 75-80 μE) at 26° C. After 4 weeks of incubation the cotyledons are transferred to fresh SB1 medium. After an additional two weeks, globular stage somatic embryos that exhibit proliferative areas are excised and transferred to FN Lite liquid medium (Samoylov, V. M., D. M. Tucker, and W. A. Parrott (1998) Soybean [Glycine max (L.) Merrill] embryogenic cultures: the role of sucrose and total nitrogen content on proliferation. In Vitro Cell Dev. Biol-Plant 34:8-13). About 10 to 12 small clusters of somatic embryos are placed in 250 ml flasks containing 35 ml of SB172 medium. The soybean embryogenic suspension cultures are maintained in 35 mL liquid media on a rotary shaker, 150 rpm, at 26° C. with florescent lights (20 μE) on a 16:8 hour day/night schedule. Cultures are sub-cultured every two weeks by inoculating approximately 35 mg of tissue into 35 mL of liquid medium.

Soybean embryogenic suspension cultures are then transformed using particle gun bombardment (Klein, et al., (1987) Nature (London) 327:70, U.S. Pat. No. 4,945,050). A BioRad Biolistic™ PDS1000/HE instrument is used for these transformations. A selectable marker gene which is used to facilitate soybean transformation is a chimeric gene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens.

To 50 μL of a 60 mg/mL 1 μm gold particle suspension is added (in order): 5 μL DNA (1 μg/μL), 20 μl spermidine (0.1 M), and 50 μL CaCl₂ (2.5 M). The particle preparation is agitated for three minutes, spun in a microfuge for 10 seconds and the supernatant removed. The DNA-coated particles are washed once in 400 μL 70% ethanol and resuspended in 40 μL of anhydrous ethanol. The DNA/particle suspension is sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. Membrane rupture pressure is set at 1100 psi and the chamber is evacuated to a vacuum of 28 inches mercury. The tissue is placed approximately 8 cm away from the retaining screen, and is bombarded three times. Following bombardment, the tissue is divided in half and placed back into 35 ml of FN Lite medium.

Five to seven days after bombardment, the liquid medium is exchanged with fresh medium. Eleven days post bombardment the medium is exchanged with fresh medium containing 50 mg/mL hygromycin. This selective medium is refreshed weekly. Seven to eight weeks post bombardment, green, transformed tissue is observed growing from untransformed, necrotic embryogenic clusters. Isolated green tissue is removed and inoculated into individual flasks to generate new, clonally propagated, transformed embryogenic suspension cultures. Each new line is treated as an independent transformation event. These suspensions are then subcultured and maintained as clusters of immature embryos, or tissue is regenerated into whole plants by maturation and germination of individual embryos.

Two different genotypes are used in these experiments: 92B91 and 93B82. Samples of tissue are either bombarded with the hygromycin resistance gene alone or with a 1:1 mixture of the hygromycin resistance gene and the CHD-DR construct. Embryogenic cultures generated from 92B91 generally produce transformation events while cultures from 93B82 are much more difficult to transform. For both genotypes, the CHD-DR construct resulted in increased transformation frequencies.

### Example 12

### Use of Antibodies Raised Against CHD to Transiently Stimulate Embryogenesis and Enhance Transformation

Antibodies directed against CHD can also be used to mitigate CHD's activity, thus stimulating somatic embryogenic growth. Genes encoding single chain antibodies expressed behind a suitable promoter, for example the ubiquitin promoter, could be used in such a fashion. Transient expression of an anti-CHD antibody could temporarily disrupt normal CHD function and thus stimulate somatic embryogenic growth. Alternatively, antibodies raised against CHD could be purified and used for direct introduction into maize cells. The antibody is introduced into maize cells using physical methods such as microinjection, bombardment, electroporation or silica fiber methods.

Alternatively, single chain anti-CHD is delivered from Agrobacterium tumefaciens into plant cells in the form of fusions to Agrobacterium virulence proteins (see, co-pending applications U.S. Ser. No. 09/316,914 filed May 19, 1999 and 09/570,319 filed May 12, 2000). Fusions are constructed between the anti-CHD single chain antibody and bacterial virulence proteins such as VirE2, VirD2, or VirF which are known to be delivered directly into plant cells. Fusion's are constructed to retain both those properties of bacterial virulence proteins required to mediate delivery into plant cells and the anti-CHD activity required for stimulating somatic embryogenic growth and enhancing transformation. This method ensures a high frequency of simultaneous co-delivery of T-DNA and functional anti-CHD protein into the same host cell. Direct delivery of anti-CHD antibodies using physical methods such as particle bombardment can also be used to inhibit CHD activity and transiently stimulate somatic embryogenic growth.

### Example 13

### Use Dominant-Negative Mutagenized CHD Gene to Transiently Stimulate Embryogenesis and Enhance Transformation

Using directed mutagenesis to disrupt critical functional domains within the CHD gene will create a dominant negative mutant. For example, single amino-acid changes in the helicase/ATPase motifs IV and VI will abolish ATPase function in this protein. In Arabidopsis, the nucleotide sequence can be modified to encode an altered amino-acid sequence, either changing LLRRVKK to LLRKVKK or changing AMARAHR to AMAKAHR. In either amino-acid stretch, changing the central arginine to a lysine or alanine residue completely destroys ATPase function in this protein. These sequences tend to be highly conserved, so altering the maize gene (or any other plant CHD gene) should have a similar effect. When such an altered CHD gene is over-expressed in the plant cell, it acts as a dominant-negative resulting in a reduction of endogenous CHD activity (and in some cases can result in essentially down-regulating CHD to the point where there is no activity).

Deletion or domain swapping techniques can also be employed to create a dominant negative mutant. For example, one of the transcriptional repression activities of CHD is achieved through deacetylation of histones. In mammalian system, CHD3/CHD4 binds to histone deacetylase through zinc-finger motif that is present in the N-terminal of the protein. Deletion of the zinc-finger motif, i.e. CQACGESTNLVSCNTCTYAFHAKCL of Arabidopsis CHD3, in this protein will change the accessibility to the histones and result in a reduction of the nucleosome remodeling activity of this protein and lead to a release of the transgenic cell from transcriptional repression.

Transient overexpression of such a dominant-negative CHD construct will result in depressed CHD activity in the transiently expressing plant cells. Genes and/or pathways normally suppressed by CHD will be transiently activated. Such a stimulation in cells receiving the foreign DNA will result in increased growth, and in species such as corn in which growth of transgenic cell clusters relative to wild-type (non-transformed) cells can be limiting, this growth stimulation will translate into increased recovery of transformants (i.e. increased transformation frequency).

### Example 14

### Increase of Oil Content in Crop Seed by Co-Suppression of CHD Gene

Expression cassettes suppressing CHD expression in seeds can easily be constructed. For example, maize oleosin promoter, or gamma-zein promoter can be used to co-suppress CHD in seed only. Transgenic seeds can be obtained by either Agrobacteria transformation or particle gun methods as discussed above. Repression of CHD expression in seed will lead to expression of many embryonic genes and change the cell differentiation. This may increase oil accumulation in endosperm or increase embryo size. Oil content in embryo and endosperm can be determined easily by hexane extraction.

What is claimed is:

1. An isolated polynucleotide selected from the group consisting of:
(a) a polynucleotide of SEQ ID NO: 42;
(b) a polynucleotide encoding the polypeptide of SEQ ID NO: 43;
(c) a polynucleotide comprising at least 75 contiguous bases of the coding region of SEQ ID NO: 42, said coding region being bases 343 to 4332;
(d) a polynucleotide having a sequence at least 90% identical to the coding region of SEQ ID NO: 42, said coding region being bases 343 to 4332, and wherein the % identity is determined by GAP10 analysis using default parameters;
(e) a polynucleotide at least 75 nucleotides in length which hybridizes under high stringency conditions to a polynucleotide of SEQ ID NO: 42; and
(f) a polynucleotide complementary to the full length of a polynucleotide of (a) through (d).

2. An expression cassette comprising at least one polynucleotide of claim 1 operably linked to a promoter.

3. A host cell comprising at least one expression cassette of claim 2.

4. The host cell of claim 3, wherein said host cell is a plant cell.

5. A transgenic plant comprising at least one expression cassette of claim 2.

6. The expression cassette of claim 2 wherein said promoter drives tissue-preferred expression of the operably-linked polynucleotide.

7. The expression cassette of claim 6 wherein said promoter preferentially drives expression in nucellar tissue.

8. The transgenic plant of claim 5 wherein said plant is a monocot.

9. The transgenic plant of claim 8 wherein said monocot is maize, wheat, rice, barley, sorghum, or rye.

10. The transgenic plant of claim 5 wherein said plant is a dicot.

11. The transgenic plant of claim 10 wherein said dicot is soybean, Brassica, sunflower, cotton, Arabidopsis, or alfalfa.

12. A transgenic seed from the transgenic plant of claim 5.

13. An isolated protein having CHD activity comprising a member selected from the group consisting of:
(a) a polypeptide at least 90% identical to SEQ ID NO: 43, wherein the % sequence identity is based on the entire length of SEQ ID NO: 43 and is determined by GAP 10 analysis using default parameters; and
(b) a polypeptide having the sequence set forth in SEQ ID NO: 43.

14. A method for reducing CHD activity in a plant, comprising:
(a) transforming a plant cell with a CHD-silencing construct;
(b) regenerating a plant from said transformed plant cell; and
(c) measuring reduction in CHD activity of the transformed plant as compared to CHD activity of a corresponding non-transformed plant.

15. The method of claim 14, wherein the CHD-silencing construct comprises a CHD silencing hairpin.

16. The method of claim 14, wherein the plant cell is from a monocot.

17. The method of claim 16, wherein the monocot is maize, wheat, rice, barley, sorghum, or rye.

18. The method of claim 14, wherein the plant cell is from a dicot.

19. The method of claim 18, wherein the dicot is soybean, Brassica, sunflower, cotton, Arabidopsis, or alfalfa.

20. A plant produced by the method of claim 14.


SEQUENCE LISTING (43 sequences)

SEQ ID NO: 1 - DNA, 1874 bp, Zea mays
  CDS (3)...(1655)
acgagaatgatgaatctcgccaaattcattatgacgaagctgcaattgagaggttgttagaccgtgatcaagttgacggtgatgaatctgtggaagatgaagaagaagatggattcttaaaaggattcaaggttgcaaactttgaatatatcgatgaggcaaaggctcaggcagaaaaagaggaggcacggagaaaggctgcagctgaggctgaaaattctgaaagaaactactgggatgaactattgaaggatagatatgatgtacagaaagttgaagaacatactgctatgggaaaagggaaaagaagccgcaaacagatggctgccgctgatgaagatgacattcatgatttaagttccgaagatgaggattactcattggaggatgacatttcagataatgacacaagtttgcaaggaaatatttctgggaagaggggacaatattctaagagaaaatcacgtaatgttgattctattccattgatggagggcgaaggacgtaccttgagagttcttggattcaaccatgctcaacgagcaatgttcctacagacactcaatagattcggttttcagaattatgactggaaagagtatcttcctcgtcttaaaggaaaaagtgtcgaggaaatccagagatatgctgaacttgtcatggcacatcttgttgaagaaattaatgattctgactatttttcagatggcgttccaaaggaaatgatgcgtgttgatgatgtactagtcaggatagcaaacatatcccttatcgaggagaagatggctgccacaggaccaggaaaaattacaaacatttttcctaattacttgctctatgagttccaaggcttatctggtggaagaatatggaaagcggagcatgatctactgttactgagaggcatactgaagcatggatatgcaaggtggcagtatatatcagatgacagagagaatgggctttttgaggctgcacgacgagagctgcatctcccttcggttaatgaaataattggtgctcagttgaacgaggcaaatgggaatttggaaggtgcacaggaaggccaagcgaacacaacaagcatgtcgcattacaaggagatccagagaaagatagttgagttcttgagaaagagatatcatcttatggagagagccttgaatctggaatatgctgtgataaagaaaaaaattcctgttcctgatgatattactgaacaaggtgttccagcaggacatgctccgcttattccagatatcagtgaactgttgcgggaattgcccaatcttgagccaatttctaccaatgaattgatttctgagggcacagctggtcagttacaagttccccatctctacaataagatgtgtggagtgcttgaagagagtggtgcttatgcgctcagttccttctttggagacaagtccgcatcttctactttggccaatagccttcgacagtttgaaactgtgtgtgagaatgtcgtcgaggccttacgaccacaccaaaatggtactgccagtgccatcaaagaggaattggtagatgcagccaccaaagcagcagcagcagcagctcctcaacaagattcaggccatgatgcaccgcatgggcagtcttcgacagccaaggcggacatggaaatcgatggttgatttgtaggttccagagtggcaagaaagggaatcccctctaatcattatgtatactgtggtcagaatgtccgctatatattgtaacatcaaagaaagcacctccaggcctgagggtgttactgctaatgcgtttggtttacttgtccttgtaatatgcatacacatttagaactcatgcagccattgtgtgaaaaaaaaaaaaaaaaaaaaaaaaaa

SEQ ID NO: 2 - protein, 551 aa, Zea mays
ENDESRQIHYDEAAIERLLDRDQVDGDESVEDEEEDGFLKGFKVANFEYIDEAKAQAEKEEARRKAAAEAENSERNYWDELLKDRYDVQKVEEHTAMGKGKRSRKQMAAADEDDIHDLSSEDEDYSLEDDISDNDTSLQGNISGKRGQYSKRKSRNVDSIPLMEGEGRTLRVLGFNHAQRAMFLQTLNRFGFQNYDWKEYLPRLKGKSVEEIQRYAELVMAHLVEEINDSDYFSDGVPKEMMRVDDVLVRIANISLIEEKMAATGPGKITNIFPNYLLYEFQGLSGGRIWKAEHDLLLLRGILKHGYARWQYISDDRENGLFEAARRELHLPSVNEIIGAQLNEANGNLEGAQEGQANTTSMSHYKEIQRKIVEFLRKRYHLMERALNLEYAVIKKKIPVPDDITEQGVPAGHAPLIPDISELLRELPNLEPISTNELISEGTAGQLQVPHLYNKMCGVLEESGAYALSSFFGDKSASSTLANSLRQFETVCENVVEALRPHQNGTASAIKEELVDAATKAAAAAAPQQDSGHDAPHGQSSTAKADMEIDG

SEQ ID NO: 3 - DNA, 21 bp, Artificial Sequence
  primer
acgagaatgatgaatctcgcc

SEQ ID NO: 4 - DNA, 23 bp, Artificial Sequence
  primer
tcaaccatcgatttccatgtccg

SEQ ID NO: 5 - DNA, 941 bp, Zea mays
  CDS (48)...(938)
gtcgacccacgcgtccgcaggattctgggaagctccacaccttggatatgctactacgacgccttcgagctgaaggtcatcgtgtgcttctttttgctcagatgactaaaatgttggacattcttgaggattacatgaatttcagaaaattcaagtatttcagacttgatgggtcttcagccatctcagaccgccgtgacatggtccgagattttcagaacaggaatgacatatttgttttcttgttaagcacaagagctggggggcttggtattaatttgactgctgctgatactgttattttttatgaaattgactggaatccaacacaagaccagcaggcaatggatagaacacacagacttggtcaaacaaaggaggtaactgtgtacaggcttatatgcaaagataccattgaggagaaaatattgcaaagagcaaagcagaaaaatgcagtgcaagagttagttatgaaggggaaacatgtccaagacgatcatttgatgagacaagaggatgttgtttcattacttattgatgacacacagattgcacacaagttgaaagaaatatccatgcaggcgaaggatcgacaaaagaggagacgagcgaagggcatcaaggttgacaaagaaggagatttgacgctcgaagacttggatgatgctactgcagaagctgtagatcaagacaaaacgaccagcaaaaagaaaaagagctcccacaagaaacatacgaatactcatgataatgacaatatagacaagaatggagaggccgatgtgggagatcatccggggagtagtaacacagaaaacgaacagatgcccgaatcaagacctaaaagatcaaaaaggctgatgaagagcattactgatgacaaggaactagctgctgctgcggatcatgagaaaccggtaaatgaagcggaaaatcactga

SEQ ID NO: 6 - protein, 297 aa, Zea mays
MLLRRLRAEGHRVLLFAQMTKMLDILEDYMNFRKFKYFRLDGSSAISDRRDMVRDFQNRNDIFVFLLSTRAGGLGINLTAADTVIFYEIDWNPTQDQQAMDRTHRLGQTKEVTVYRLICKDTIEEKILQRAKQKNAVQELVMKGKHVQDDHLMRQEDVVSLLIDDTQIAHKLKEISMQAKDRQKRRRAKGIKVDKEGDLTLEDLDDATAEAVDQDKTTSKKKKSSHKKHTNTHDNDNIDKNGEADVGDHPGSSNTENEQMPESRPKRSKRLMKSITDDKELAAAADHEKPVNEAENH

SEQ ID NO: 7 - DNA, 22 bp, Artificial Sequence
  primer
aagctccacaccttggatatgc

SEQ ID NO: 8 - DNA, 23 bp, Artificial Sequence
  primer
tcatgggctcagtgattttccgc

SEQ ID NO: 9 - DNA, 1913 bp, Zea mays
  CDS (3)...(1910)
cacacttaataattgctccaaaagcagtattaccaaattggtctaacgaattcaaaacctgggctcccagtattgggacaattctgtatgatggtcgtccagaagagaggaagcttttaagggaaaagaattttgatggattgcaatttaatgttttgctcacgcattatgacttgatactgaaagataagaagttcctaaagaaagttcactggcattatttgattgttgatgaaggacatcgtctgaaaaatcatgaatgtgctcttgctcgcacactagtttcaggatatcagatccgccgcagactacttttaactggcactccaatccaaaatagcctacaagaactgtggtctttgcttaactttattctgcccaatatttttaattcatctcagaattttgaggaatggtttaatgcaccatttgcatgtgatgttagtcttaatgatgaggaacagctattaatcatacatcgtctgcatcaagttttgcgtccatttttgctgaggaggaaaaaagatgaagtggaaaaatatctccctgtcaaaacacaagtaattctcaagtgtgatatgtctgcttggcaaaaagcatactatgaacaagtcacaagcagggaaaaggttgcactaggatttgggctcagatcaaaggctctgcagaatctgtcaatgcaacttaggaaatgttgcaaccacccctatctatttgtagagcactacaacatgtaccagcgggaggaaattgttagagcatcagggaagtttgaattgcttgatcgtctacttccaaaactgcagagagctggtcacagggttctgcttttctctcagatgacgaaactgcttgatgttttagaaatatatttgcaaatgtacaatttcaagtacatgaggcttgatggatccacgaagactgaagaacgagggaggttactggcagattttaataagaaggattcggaatatttcatgtttctcctcagcacacgtgctggaggacttgggttgaacttgcagacggcggacactgtcattatatttgatagtgactggaaccctcaaatggaccaacaagctgaagaccgtgcccatcgtataggcagaagaatgaagtgcgtgtgtttgttcttgttagtgtcggctccattgaagaagagatcctggaccgtgcaaaacaaaagatgggtatcgatgcaaaagttactccaggctgggttgtttaacacaacttccacagcacaggacagacgagcattgctgcaggagatccttaggagggggacaagctcgctgggaacagatatccccagtgagcgcgagataaatcgtttggctgcacgaactgatgaagaattctggttgtttgagaagatggatgaagaaaggaggcttagagaaaactacaaatctagacttatggatgggaatgaggttccagattgggtattcgccaacaatgatttacccaagagaaccgtggcagatgagttccagaatataatggtcggtgcgaagcgacgtagaaaggaggttgtctattcagactctttcggtgatcagtggatgaaatccgatgagggatttgaagacattccaaaggcgactcagaggtcgaagaagactgcttactcatctgacatccaagttgagtttagtgaaaggaggaaaagacctaggtctgtagaaaacagcgcagacggtgtgagcaacccgacgtggacgcctgacaaaggaagggctggagtttcatcatacagcaaggacgagactgaagatgatggcgaagacgaagtcattactagcggcttacaaaagggaaacagtttcacatggaataccctaggaagaagaaggtcaagccacttcagttcgtcatcggactcgagagggcgcccaacattctaa

SEQ ID NO: 10 - protein, 636 aa, Zea mays
HLIIAPKAVLPNWSNEFKTWAPSIGTILYDGRPEERKLLREKNFDGLQFNVLLTHYDLILKDKKFLKKVHWHYLIVDEGHRLKNHECALARTLVSGYQIRRRLLLTGTPIQNSLQELWSLLNFILPNIFNSSQNFEEWFNAPFACDVSLNDEEQLLIIHRLHQVLRPFLLRRKKDEVEKYLPVKTQVILKCDMSAWQKAYYEQVTSREKVALGFGLRSKALQNLSMQLRKCCNHPYLFVEHYNMYQREEIVRASGKFELLDRLLPKLQRAGHRVLLFSQMTKLLDVLEIYLQMYNFKYMRLDGSTKTEERGRLLADFNKKDSEYFMFLLSTRAGGLGLNLQTADTVIIFDSDWNPQMDQQAEDRAHRIGRRMKCVCLFLLVSAPLKKRSWTVQNKRWVSMQKLLQAGLFNTTSTAQDRRALLQEILRRGTSSLGTDIPSEREINRLAARTDEEFWLFEKMDEERRLRENYKSRLMDGNEVPDWVFANNDLPKRTVADEFQNIMVGAKRRRKEVVYSDSFGDQWMKSDEGFEDIPKATQRSKKTAYSSDIQVEFSERRKRPRSVENSADGVSNPTWTPDKGRAGVSSYSKDETEDDGEDEVITSGLQKGNSFTWNTLGRRRSSHFSSSSDSRGRPTF

SEQ ID NO: 11 - DNA, 23 bp, Artificial Sequence
  primer
cgaattcaaaacctgggctccca

SEQ ID NO: 12 - DNA, 21 bp, Artificial Sequence
  primer
ttagaatgttgggcgccctct

SEQ ID NO: 13 - DNA, 1463 bp, Zea mays
  CDS (3)...(1460)
  misc_feature 429, 1443: n = A,T,C or G
gtcgacccacgcgtccgccagaagagcggaaccatataagggacaatttgctgcaacctgggaaatttgatgtgtgtgtgactagttttgaaatggcaatcaaagaaaaatctgcgttgaggcgcttcagctggcgctacataatcattgatgaagctcaccggataaaaaatgaaaattctcttctatcaaagactatgaggatttacaacactaattatcgtctcctcatcacaggcactccactccagaataatctccatgagctctgggctctcctcaatttcttgctacctgaaatatttagctctgcggagacctttgatgaatggtttcaaatatctggggaaaatgatcaacaggaggtggtgcagcagcttcataaggttcttcgcccattccttcttaggaggctcaagtctgatgtanaaaagggcctacctccaaagaaagaaacaattcttaaagttggaatgtctcagatgcaaaagcagtactatcgtgctctgcttcagaaggatttggaggttattaatgctggtggtgaacgcaagcgattgcttaacattgccatgcagttgcgcaagtgctgcaaccatccatatttattccaaggagctgaacctgggccaccctacacaactggtgaacatctaattgagaatgcaggaaaaatggttctacttgataaattgctgcccaagctaaaggagcgtgattccagagtccttattttttcacagatgaccaggcttttggatatcttggaagattatcttatgtataggggatatcagtattgtcgaattgatggaaatacaggtggagaagatcgtgatgcatccattgaagccttcaatagtccaggaagtgagaagtttgttttcttactttcaactagggcaggtggccttggtatcaacttggccactgctgatgttgtggttctctatgacagcgattggaatccccaagctgatctgcaagctcaggaccgtgcacatagaataggtcaaaaagaaagaagttcaagtgttccgcttttgcacttgagttcaactattgaggaaaaggtgattgagagagcatataagaagctagcattggatgctttggttattcagcaaggacgattggcagagcagaaaactgtcaataaggatgatcttctgcaaatggtgcggtttggtgctgaaatggttttcagttctaaggacagcacaataactgatgaggacattgaccgtattatagctaaaggagaggagacaacagcagaacttgatgcgaaaatgaaaaagttcactgaggatgccatcaaatttaagatggatgataatgctgaattgtatgacttcgatgatgagaaggatgaaaacaaggttgatttcaagaaacttgttagtgataactggattgagccacctagaagagaaaggaagngaaactactctgagtcttga

SEQ ID NO: 14 - protein, 486 aa, Zea mays
  VARIANT 143, 481: Xaa = Any Amino Acid
RPTRPPEERNHIRDNLLQPGKFDVCVTSFEMAIKEKSALRRFSWRYIIIDEAHRIKNENSLLSKTMRIYNTNYRLLITGTPLQNNLHELWALLNFLLPEIFSSAETFDEWFQISGENDQQEVVQQLHKVLRPFLLRRLKSDVXKGLPPKKETILKVGMSQMQKQYYRALLQKDLEVINAGGERKRLLNIAMQLRKCCNHPYLFQGAEPGPPYTTGEHLIENAGKMVLLDKLLPKLKERDSRVLIFSQMTRLLDILEDYLMYRGYQYCRIDGNTGGEDRDASIEAFNSPGSEKFVFLLSTRAGGLGINLATADVVVLYDSDWNPQADLQAQDRAHRIGQKERSSSVPLLHLSSTIEEKVIERAYKKLALDALVIQQGRLAEQKTVNKDDLLQMVRFGAEMVFSSKDSTITDEDIDRIIAKGEETTAELDAKMKKFTEDAIKFKMDDNAELYDFDDEKDENKVDFKKLVSDNWIEPPRRERKXNYSES

SEQ ID NO: 15 - DNA, 23 bp, Artificial Sequence
  primer
ccagaagagcggaaccatataag

SEQ ID NO: 16 - DNA, 23 bp, Artificial Sequence
  primer
ctcttctaggtggctcaatccag

SEQ ID NO: 17 - DNA, 1645 bp, Zea mays
  CDS (2)...(1642)
  misc_feature 260: n = A,T,C or G
agcagatgggagaagatacatgatccgccggagactacttttaacaggcactcctatccaaaacagcctgcaagagctctggtctttgcttaacttcatcctgcccaatatttttaattcatcccagaattttgaggaatggtttaatgcaccatttgcatgtgatgtcagtcttaatgatgaggaacaactactaatcatacatcgtttgcatcaagttttgcgtccattcttgctgaggaggaagaaagatgaagtanagaaatatctccctgtgaaaacacaagtaattctcaagtgtgacatgtctgcttggcaaaaagcatactacgaacaagtcacaagcagggaaaaggttgcgctaggatatgggatcagaaagaaggctctgcaaaatctgtcaatgcaacttaggaagtgttgcaatcatccctacctattcgtagagcattataacatgtaccaacgggaggaaatagttagagcatccggaaagtttgaattgcttgatcgtctacttccgaaattgcagagagctggtcacagggttttacttttctctcagatgacaaaattgcttgacgttttagaaatatatttgcagatgtacaatttcaagtacatgaggcttgatggatccacaaagactgaagaacgtgggaggttactggcagattttaataagaagaattcagaatatttcatgtttcttctcagcacacgagccggaggtcttggattgaacttgcagactgcagacaccgtcattatctttgatagtgactggaaccctcagatggaccaacaagctgaggaccgtgcccatcgtatagggcaaaagaacgaagtacgtgtgtttgttcttgttagcgttggttcaattgaagaagagatattggatcgtgcgaaacagaagatgggtattgatgcaaaagtaatccaggctgggttgtttaacacgacctccacagcacaggacaggcgagcattgctgcaggagatactcaggagaggaacaagctcactgggaacggatatccccagtgaacgtgagataaaccgcttggctgctcgaaacgatgaagaattccggttgtttgagaagatggatgaagaaaggaggctaaaggagaactacaaatctagacttatggatggaaatgaggtcccagattgggtgtttgccaatgataatgaaaccttacgcaagaaaaccgtggcagatgaattccggaatataattgttggttcaaagagacgtagaaaggaggttgtctattcggactcttttggtgatcagtggatgaaatccgacgagggatttgaagagattgcaaagatgactccaagggtgaagcgaactgcttattcgcctgacattcaagttgagtacaatgaaaggaggaaaaggcccaagtctgtggaaaacagcgcagatggcgcaagcaacccaacacggacacccgacaaaggaagggctggagtttcatcatacagcaaggatgagaccgaagatgatggtgaagacgaagtcatcaccagtggcttacagaagggtaacagtttcacatggaagacccttggaagaaaaaggtcaagccacttaagttcgtcgtcggactcaaaagggcgaccatcattctaa

SEQ ID NO: 18 - protein, 547 aa, Zea mays
  VARIANT 87: Xaa = Any Amino Acid
ADGRRYMIRRRLLLTGTPIQNSLQELWSLLNFILPNIFNSSQNFEEWFNAPFACDVSLNDEEQLLIIHRLHQVLRPFLLRRKKDEVXKYLPVKTQVILKCDMSAWQKAYYEQVTSREKVALGYGIRKKALQNLSMQLRKCCNHPYLFVEHYNMYQREEIVRASGKFELLDRLLPKLQRAGHRVLLFSQMTKLLDVLEIYLQMYNFKYMRLDGSTKTEERGRLLADFNKKNSEYFMFLLSTRAGGLGLNLQTADTVIIFDSDWNPQMDQQAEDRAHRIGQKNEVRVFVLVSVGSIEEEILDRAKQKMGIDAKVIQAGLFNTTSTAQDRRALLQEILRRGTSSLGTDIPSEREINRLAARNDEEFRLFEKMDEERRLKENYKSRLMDGNEVPDWVFANDNETLRKKTVADEFRNIIVGSKRRRKEVVYSDSFGDQWMKSDEGFEEIAKMTPRVKRTAYSPDIQVEYNERRKRPKSVENSADGASNPTRTPDKGRAGVSSYSKDETEDDGEDEVITSGLQKGNSFTWKTLGRKRSSHLSSSSDSKGRPSF

SEQ ID NO: 19 - DNA, 23 bp, Artificial Sequence
  primer
acaggcactcctatccaaaacag

SEQ ID NO: 20 - DNA, 23 bp, Artificial Sequence
  primer
gaatgatggtcgcccttttgagt

SEQ ID NO: 21 - DNA, 514 bp, Glycine max
  CDS (6)...(514)
  misc_feature 3, 25, 27, 114: n = A,T,C or G
ccntaaatttcttgttacccaaacntnatcaatttcatccaggaggacttctctcaaatggtttaataagccatttgagagtgcttggagatagctcgcctgatgaagctttantgtccgaggaggagaatctcttgattataaatcgtctgcaccaagttttgagaccatttgtacttaggaggctgaaacacaaggttgaaaatgagttgcctgagaagattgagagactaataagatgtgaggcctcatcatatcaaaaacttttgatgaagagggtggaagaaaatcttggttctattggcaattcaaaggctcgatcagtacacaactctgtcatggagcttcgtaatatatgcaatcatccatatctcagtcagcttcatgcagaggaggtggataacttcatacctaaacattatctgccaccaattattagactttgtgggaagcttgagatgttggaccgtttattgccaaaattgaaggcgacagatcatcgggttcttttctt

SEQ ID NO: 22 - protein, 169 aa, Glycine max
  VARIANT 7, 8, 37: Xaa = Any Amino Acid
NFLLPKXXQFHPGGLLSNGLISHLRVLGDSSPDEALXSEEENLLIINRLHQVLRPFVLRRLKHKVENELPEKIERLIRCEASSYQKLLMKRVEENLGSIGNSKARSVHNSVMELRNICNHPYLSQLHAEEVDNFIPKHYLPPIIRLCGKLEMLDRLLPKLKATDHRVLF

SEQ ID NO: 23 - DNA, 23 bp, Artificial Sequence
  primer
aacccgatgatctgtcgccttca

SEQ ID NO: 24 - DNA, 23 bp, Artificial Sequence
  primer
tcatccaggaggacttctctcaa

SEQ ID NO: 25 - DNA, 403 bp, Glycine max
  CDS (221)...(403)
  misc_feature 8, 10, 15, 31, 48, 62, 79, 181, 194: n = A,T,C or G
tgaatatntncttgnttttaatttatgcgantaaggatttgtgcattnggagattagtgtcnatgaatcaagtgattgntattttatttcatgtgtcacccagccatattggcagatgaaatgggtcttggcaaaacagttcaggtacgtattctgttttttattattttaatatgtttcntaatttgtttgtnttcctaatcctttacttttcaagtaagaaatgccatatgttcttgtcttccaggccatcacatatttaactttgctgaaacacttgcacaatgattctggtccacatcttatagtatgtcctgcttctgttctggaaaactgggaaagggaattaaaaaggtggtgtccatccttttctgttcttcaataccatggggccggacgtgca

SEQ ID NO: 26 - protein, 61 aa, Glycine max
EMPYVLVFQAITYLTLLKHLHNDSGPHLIVCPASVLENWERELKRWCPSFSVLQYHGAGRA

SEQ ID NO: 27 - DNA, 23 bp, Artificial Sequence
  primer
gccccatggtattgaagaacaga

SEQ ID NO: 28 - DNA, 25 bp, Artificial Sequence
  primer
attttatttcatgtgtcacccagcc

SEQ ID NO: 29 - DNA, 522 bp, Oryza sativa
  CDS (1)...(522)
  misc_feature 463, 510: n = A,T,C or G
gtttctgggaggaaggctcagtattctaagaaaaactcacgtaatgtagattcactccctttgatggagggtgaagggcgtgctttaaaagtttatggattcaatcacgttcaacgaacacaattcctacagacactcatgaggtatggttttcagaactatgattggaaagagtatcttcctcgtttgaaggggaaaagtgttgaggaaattcagagatatggtgagcttgtcatggcccatcttgtagaggacacaaatgactcaccaacctatgcagatggtgtgccgaagaaatgcgtgctgatgagacattggtcaggctagccaaaatatcacttgtggaggagaaggtggtgcatggagcaaggaaaattacaaaactcttccccaactacttgatgtatgaatttactggcttatcaggtggaagaatatggaaaggggaacatgatctactgtnactgaagcataataagcacgggttgccagtggcatacatatcagatncagagatacggg

SEQ ID NO: 30 - protein, 171 aa, Oryza sativa
  VARIANT 153, 167: Xaa = Any Amino Acid
VSGRKAQYSKKNSRNVDSLPLMEGEGRALKVYGFNHVQRTQFLQTLMRYGFQNYDWKEYLPRLKGKSVEEIQRYGELVMAHLVEDTNDSPTYADGVPKKCVLMRHWSGPKYHLWRRRWCMEQGKLQNSSPTTCMNLLAYQVEEYGKGNMIYCXSIISTGCQWHTYQXQRYG

SEQ ID NO: 31 - DNA, 23 bp, Artificial Sequence
  primer
gtttctgggaggaaggctcagta

SEQ ID NO: 32 - DNA, 23 bp, Artificial Sequence
  primer
tatgtatgccactggcaacccgt

SEQ ID NO: 33 - DNA, 510 bp, Oryza sativa
  CDS (2)...(510)
cttacaggatttcgggggaggtggctgcggctgtttggagcggaggggtttaatagctacagcatgtgacgttgatactctaatgatgaaggagcggagctctttatgtgaaagtgcggcagatggaagttgggttttgaaatacaaaaggaaacggagcaagctaacagttagtccatcaagtgagcatgatgcttcctcaccaatactggattctcaaatgaacaatggctccatcaaaaagaagatcaaacatgacactaacatttctccatcaaccaagaagataagaggacatgacgggtacttctacgagtgtgtagaatgtgatctcggtggcaatttgctgtgctgtgatagctgtccacgaacataccacttggaatgtcttaatcctcctctcaagcgtgcaccacctggaaattggcaatgcccaagatgtcgtacaaaaaaagttagcttgaagctcttaaacaatgctgatgctgacacctcctaaacgtgaaagaa

SEQ ID NO: 34 - protein, 167 aa, Oryza sativa
LQDFGGGGCGCLERRGLIATACDVDTLMMKERSSLCESAADGSWVLKYKRKRSKLTVSPSSEHDASSPILDSQMNNGSIKKKIKHDTNISPSTKKIRGHDGYFYECVECDLGGNLLCCDSCPRTYHLECLNPPLKRAPPGNWQCPRCRTKKVSLKLLNNADADTSTK

SEQ ID NO: 35 - DNA, 23 bp, Artificial Sequence
  primer
cttacaggatttcgggggaggtg

SEQ ID NO: 36 - DNA, 23 bp, Artificial Sequence
  primer
ctttcacgtttaggaggtgtcag

SEQ ID NO: 37 - DNA, 667 bp, Triticum aestivum
  CDS (2)...(667)
  misc_feature 543, 566, 568, 575, 597, 608, 621, 647, 657, 659, 667: n = A,T,C or G
gttgactggaaccccattacagaacaacattggtgaaatgtataatttgttgaacttcctacagcctgcttctttcccttctctagcatcatttgaggagaagtttaatgaacttgcaacagcagagaaagtggaggagctgaagaaactggtagcaccacatatgcttcgaaggctgaaaaaagatgcaatgaaaaatatccccccgaagacagagcgaatggtgcctgtcgaactgacatcaatccaggctgaatactaccgtgctatgcttacaaagaactaccaagtactgcgtaataccggaaaaggtggtgctcatcagtcattgctcaatatagtaatgcagcttcggaaatttgcaaccatccatatcttatcctgggaactgaacccgaatcaagttcaccagatttttgcatgaaatgagaataaaggctcaacaaattaactttgttgcattctatgctcaaagtgttacacagtgatgggcatcgtgttctaattttccagatgactaaactcttgacatcccgaagatancgacccggaattggcataaacantnaaagagnaatggtcgtgtcgtgggtgacncaagcacatancttcaacagaaanacgtttgatttgtacaacggcatgcntgtattgacnanacgtacan

SEQ ID NO: 38 - protein, 214 aa, Triticum aestivum
  VARIANT 176, 183, 186, 192, 196, 200, 209, 211, 212, 214: Xaa = Any Amino Acid
LTGTPLQNNIGEMYNLLNFLQPASFPSLASFEEKFNELATAEKVEELKKLVAPHMLRRLKKDAMKNIPPKTERMVPVELTSIQAEYYRAMLTKNYQVLRNTGKGGAHQSLLNIVMQLRKFATIHILSWELNPNQVHQIFANENKGSTNLCCILCSKCYTVMGIVFFSRLNSHPEDXDPELATXKEXWSCRGXKHIXSTEXRLICTTACXYXXVX

SEQ ID NO: 39 - DNA, 23 bp, Artificial Sequence
  primer
gttgactggaaccccattacaga

SEQ ID NO: 40 - DNA, 23 bp, Artificial Sequence
  primer
catgccgttgtacaaatcaaacg

SEQ ID NO: 41 - DNA, 12561 bp, Zea mays
  misc_feature (1)...(12561): Zmpkl genomic sequence
  misc_feature 11757, 11808, 11859: n = A,T,C or G
atactgtaatcatctatgacaggtgaaaaatctctctgtttagacaaaacagttaacataatggattcgcttcaatttctcacctatgtatgtacagtgattggaacccacatgcggatttgcaagctatggcaagagctcatcgcttaggacagactagtaaggtattttaccttacactttatattgtataaaaaaacagattttcaataagttttgtggtgattttataattttcatctgtttttcttttaggtgatgatatacaggcttgttagccgaggtacaattgaggagcgaatgatgcagcttacaaaaaagaagattttattggagcacttagttgttggtcgactcaccaaagctaataatgtcaatcaggtatgttgactactttttaatggtgaattttgtaaaccatcaacttaggttgatcttttatggcctaagctatttatgaattcatttatggattgagggttgagtagttacatgttactccctccattttttatatttgtggtgttttagttcaaaaataaactaacgggtgacaaatattcgagaacggaggtagtactagtaccttctgtctgggatgacatgaaatgaatgtagcatctgttagtatcatgtccatttctttgtgttacattttacaaggcttaaaaccttacacatattgccggagttggtgactatttagtcttatctgtaaatttagttgtttctcttgatgtcaatagcaatttatggttgtatgagatttcgtgggtttgttagcatgtgtgccatataggtttagctccgctgatgtgttatgcacttataattcagacccattttggagctgtgatgtgatacacaatgctagttgttaaggccccatttgtttgtttcatattcataatctatgtgcatgcattagtccagatcaggctgggtgatctggtgggcattggtacatgcatggctgaataagagtttgatgaccaagggtaaggcaccccacatctactcttggctggccaactgggtagtatctgctagttatcatggaaataaggttggcaacctctttaaggttgtcttcaagaactaaacataaaaagaatgccatgaagatggaacccaggatctcaggacctatttgtcgtaagatttctgaatctttgctggagctggcaacgggctggttgctggtctcatccctacattatagttttgtgtatgtttcttccagactttcaacacttctcaacttctagaacatgtaaccggcatgtacaaacagaacaatttagaacagttcaaagtgcatgtccttactgcgatgggaccacacacttctggtctctttaggacttgattgatttaatgcagattaataattaacccaatttgctcttctgacctgctagtgagcccttcattggtttttaagtactaaataataaatatgtgtctttgcctattttaggaggagttggatgatattatacgctatggatcaaaggagctttttgaagacgagaatgatgaatctcgccaaattcattatgacgaagctgcaattgagaggtaaacacctaggcccttttttgatctcctaagataagattaatgagaacgacgtagaaaaatgagtgtgatccaagcacagattctagaatccaactatctagctaaaccttgctatacatagattctcgctatgcaaaggccataatcactatgaaaatgagatccaaacaccccttgtttttatttatctagaatgtagattctcaaaagaagggcaagcttggtgcattggtgagatctttctcattcagtcatcaagtcgtgggttcaaagcagcctctcgacatttgtgggagcctctagcactgggtctacccctttttattaacaatctagattctacatcatcatgtagaatcactatgataatgagatctaaacatggccttaagcccctaacttcaccttgggaagttatttcgtgcattaagcatgcctttttggtgtgaagttgtttaaagtattaccagatttatgtaacaatttacaaatgtattggaatctggagaccattttgatcatggatgcaaatctagaattctagagataattagtatgtatgccctaccaagtaggatacgatgagagattgagagtgctaagagacttttatgggaagacttagatggtatgattagagttatgcctattagtgagaagcttttcataggagatctcaacggacatgtaggtacaacaagtgtaggtttcaaggcggttcatggagggtttgaggaagagaatcccattttgtgacatgtagtagtggcaaacgctctagtcagatttgttaagtaactgtgtattccttgtaatgggatcttgcgtaatgagcataggccctataactctattataaatacagcaccgaaccctgatgttaagctaggtttagcctcctctctcccacctagccaatatggtatcaagctaggtttagcctcctctctcccacccagccgtcgccgccaccgctacagtagttgccgctgtcggcattcttcctcccctcccccttccctggtgtcggcgaccctccttcgacctccttccatcggcgcccctttcccctcgtccagccgtcgccgcagtgtctggacgcgaggcccttcctcatccgtgcgcttgggggccgctgggcttttcctccccccgcccgccagcagtcggccttgtcctcgccaccgtcggagttgccacatgagaggcattgaaagggtgaaaggaggtaatgcggtgggccctgatgttatcccaatcgaggcatggagatgtcttgggggacatagctataataggctaactaagttgttcagtctatcttccggtcaaacaagatgcttgacaagtggagaagtatattggtaccaatcgacaaaaataaggaagatattcaaagttgtagcaattaatagggaataaagttgatgagccatactacgaagctatgggagagagttatcgagcatcgagaggaaaaacgaggacctgtatgaaccaatttggtttcatgcaagctcaaccatggaagccattttcttaataagataagtactttgtgactaacataaaattttatgtggtaggctttggacaaacataaaattccaacgaagtactttgtgactaattaaggacatgtacaataatggtgtgactagtcactagtgtttgaacaagtcatgtgaacacaaatgacttactgattagagtagggctacatcaggggtcaaccttgagctcttacctttttgccttggtgatggatgaggtcacaaaggacatacaaatggatatcccttggtttatgccttgcggacgatatagcgttatttgatgaaagtcggatagagttatttgatggaagtcggataggagtaaataggaaactatagctgtggcgggagactctagagtccaaaggttttagactcaatagaactaaaactgaatacatgagatgtgtcttcggcactactacacataaggaaaacgatgttagtttgaaaggtcaagtagtgcctacgaaacgataatatacatgatagattaggggtgacaccaattgaagaaaagcttttccaacaccgattgatatggtttgaacatgtctaacagagacctctagagacaccaatgtgtagtggaattctaagtcatgatagtaatgagaagagacaagagaggcagaggaaggccaaagttgacatggaaatgggtagtagaagagatttcaaaggatgaaatataccaaagatttagccttaaataggagcgaatgggaaacaactcatccatgtgcctgaatcttgatttgtggctttattaggtttcaactctagcctagcccaacttgattgggactaataggctttgttgttgtttttgcaaacctggaaattttcatgaccatggcaataaatagcacagtttatactcaatatcaccctatacgtaagaacaacatgagctgcataattgattttgtttgtttaattccattttcttatgaaattccttcttttctcctattacagtaattcatagtatggaatctgtcttcatgcagattatgcacaacactaatccttgttgttatggccccaatttttatcttctttgttatgacttaaaactgtagtgctctatgttagaggagacaacgtaatatatctgactggactcacaggttgttagaccgtgatcaagttgacggtgatgaatctgtggaagatgaagaagaagatggattcttaaaaggattcaaggtattggggtcttctttcaattattacaagcataatgcttgaggagcttcttcattttaattatcctcttgatatttactgtggtttacattgtttagtctttttcttgtttattatgtgcacatgtatttgttaaagtgcacataatctctatttgcacaagtacacttgtgaggcagtgaggtctgactctgttatttgtatgtttgcgagtatgcatgtaatcaagccactattgatatttgataggattaacctatgcatcagattttgttgatgaatgagatattttatgtatgcattgcatgctttctcatttgaattatctcgttagttttacctgcccatctaagatatacaattgcgtagtgctgagtaaaacactatgcaaataaacaatgtttcttgttctctcattcatctgtaggcctattcttccaattcaaacatgctatgttactcatagggcaatactatttgtgtttcatttattgttgaaaatgctgcatactacaactgcctcatgactcatttttcattctaagtgtttggcacattctagcaaacaaaagagtccctttatttaacccaaagcaaaacgattctcgcttcttgtattactctatgctaggttgcaaactttgaatatatcgatgaggcaaaggctcaggcagaaaaagaggaggcacggagaaaggctgcagctgaggctgaaaattctgaaagaaactactgggatgaactattgaaggatagatatgatgtacagaaagttgaagaacatactgctatgggaaaagggaaaagaagccgcaaacaggtttaatttctaaccatttccattgttaaccttgtgacttgtgccccttttcatatcattttcccttgttttgtgatctgcattcattgctttgcggttggcaatagatgtaatttcatatttgttccttcccaaaaagaaaatcatagttgttgccttgttggcaacttacagtatttcacttgtagaatatcttttaaactcgatttaagcactgtaattgtaattaaattttaggaaatcatgcttatacaccaactaggataactgatgcaaccaatcactgttgagcgttgacaatatgcatcaaagtcatataactaatcttatcatgaagataagcatgaaattaaggaagaacgtagccattatttgttatatctttcttggtttaacggcgcagtgccattagtgcattacttcttagccagacatgacatctgctgctcagtgtagtgtaatcttcttttgcgaaggtacttctctttaaactagttttgcatgacgatgaagttgagtatagacattttgtgttagatccttcttgctgttactgtacagtatatatattagttcattattcgtcccaccccaccccaaaccaatcattgactatagcttaatgtactatgtctgtttggtaatgtattgtattgtttttatttggtcgatttaaattttagagtgaccatatacattttcagtaagagaaaagactgatgaaccttgtttcttttctgcgtcatgtgcagatggctgccgctgatgaagatgacattcatgatttaagttccgaagatgaggattactcattggaggatgacatttcagataatgacacaagtttgcaaggaaatatttctgggaagaggggccaatattctaagagaaaatcacgtaagagagcaatgtaaatacatcgcactatggactattgttacatgatgaatattctgtacttatatactttgcaaatacattgtcattaggtaatgttgattctattccattgatggagggcgaaggacgtaccttgagagttcttggattcaaccatgctcaacgagcaatgttcctacagacactcaataggttagttactgatatgcctcttgaacctgtctggtcagcgagtgagtaccttgaacctaaagtttatgtgcagatgttgagatgctattacattgcgtgataaagcaggcacacagaaatttctgtttcatttgtatctcttggccatgtgtacatttttaatcatggaatccttttttttttacaattgttggttagtgtttacatttttatcactcgtttctaatagttgtgtgctctgacctgtatctttgtcaccaaattgtaatttctggtccaattttaagcataaaatatttgcactgaggtcattggctattccctagttgtactatcagaccaacatgaacgtgaccccatgcaacattgtagattcggttttcagaattatgactggaaagagtatcttcctcgtcttaaaggaaaaagtgtcgaggaaatccagaggtatgtgaaatgtgctctccatgttttatgaccccaattattttgagttgtagaccaaatagtgaagaatctggggcatagtttcgtaagttagatggatatgatgcacaacattttacattttattgattttctcttaggcgaagggcgggcctggtgcagcggtagagcctaccgtctgtaaccggaaggtcttgggttcgagccccaacctctgcatattatgcgggtaaggcttggcgcttaaagatacccttccccagaccccgcacagtgcgagaagcctatggcactgggttcgcccttttattgattttctcttaggccccgtttgtttcccttcattttaaggaattggaatctaactgatggagtaagctattttttttataatgtaatattccataactttccaaagtttatgtataagccaatctcaaattcatggggtgagagatggaaattgattctatagatttacatgctacttttcaaatttacaacttatagcacactcttctacttgcttctctatatgataaatgtagtgtataactatctctcttatatgatttaggataatatacaaatacattacatagataaatatattaacttaatagttttatcttaaattataattattataatggaattcaattccaacgaaacaaacggggccttaggtatatagacaatgaattcaagtttgtgcttcgacgaagaattgatgagatgggtaactgggcattcctggcttctgatagtacacatatttaggcaaagtcgactgctggtagtagtgaaagtgtacacacaaatatgcttggttggactcttgctttgtttattatgttgcaaatttatataactattgcacccttgccaacaaggaaagatgtggcatcatcacatatgaagccaaattaagcaggcagtcaccaaagtcagactatatctgcagtgctagactgttagtagcaaattgagccaacagtcaccaaagctagactacctctgaagtgcgaatagcaggcggccataacctaaccctattaagttgtatgcaccaaccagttcaacccaaaagcttaagctgatggagagaggtggcaattcacttgtattctaacattctccctcacatcgaggctctcttagaccgtctccagcagttcacccatacggttatcaaaacactggttttcactgtagactatactgtttgcatatggggatgtggatgagtaagctgctggagatagccttaggtcttagacgtggaataagaacggacaacaattattttatttaattgcgctaaccaggattcgaactcaagatatctggctttgatatcatattaagtcgcatataccagccagttcaacccaaaaacttaagcttatagagaggtggacaattcacttgtattctaacaaaccccagtgctagtagcaaaatgagccaacagttaccaaagcttgcagctgaagggctagtttggcagcgctttgtggacagagcgctgcgccgctgccaaacactgttgctccatctcggaagtgctcgccctatgctccagtcgatttgcattatgggtggggagcggaaaaaatccgctctgtctgcagcatgctcctctctccacttcccgctctgctgcctgctcccctctccacccacgccgctatccaccgcatctctgcctgcacagcccctctctggctcattaacggtggcgtggaccacgagcactgggcagatccgtggtggtgataaagagggcaggagggaggccagaccctccgtggcgaggagtgctgcacttggaggatgtgggccctggcgagctggagccgaagctgatggagaagcactaccaaacactggtatcggcaggtggggagcaacctgagcgggagcctaggggagctggagccgctgggagcttcgtgacagtagtaccaaacatgatctaagtaaacgtcatatgaacatggaacaggcatcaagatgctgtccttcatatagctctatcattactttttccttgtagagcccataaccacaagaatgttgtacacccacttatgtatagggtaggcttgtataacacccatttaagtcaagtacacctcttatattcccaccaaacaacccagttttaggcaattatttttgtggagatcctcaaagtgattcatagcaataacctttgtgcaatttattttcgtatatataatatgttcttgcagatatgctgaacttgtcatggcacatcttgttgaagaaattaatgattctgactatttttcaggtaattgagcttagtaatggctactatcatttttacgcatccaattcttatcctccatatgcatgaatgcagatggcgttccaaaggaaatgatgcgtgttgatgatgtactagtcaggatagcaaacatatcccttattgaggagaaggtgcatgtggctttcatttgttatttgcatcttaacatgacttagaactcaaaagaaacttatgagcatcctgatgatgttcaatacaaacatgattgtgctgtattctttcttgcgtaaagtgcaaggattgtcgagagctgaagtttagtatttaaacttgcaccttatagaattcggttgattacaataccttgatatgattctgctatcaatgaggcaacctttgtgtactgtattctttcttacttttgtattgtggttccagatggctgccacaggaccaggaaaaattacaaacatttttcctaattacttgctctatgagttccaaggcttatctggtggaagaatatggaaagcggagcatgatctactgttactgagaggcatactgaagtacgaaactacgaatattttctttgttacaatcagctctacaaaattacccctccatgaaactagcagtggctggaacagttctggcaagagtagccttaaaaagtcgatgtttgtgtcagcagcagtggcgttgcaatgacagaactgaattgtttggctcacttgttggttgatggattattagttttttagtatagcagcagtggcttgcatcttggcccgagtggacctattgtgctcagaagttagggttaccctaacgggtatcttttcgcatagcttgatagcttccaggtggtgcctgtccacccagtgctctagtgattttggagacatcgggttcaggaactgtttaattaaggcaacaaattcaacttatcttaacgttgagtaagcaaactgaagtatcacatgcaaaccagatgagctccacatttgatttgatatctaacttaaattatttattttgattctgtttaatacttttacaattgtaatcatgtggaacagcttaatgaaaaatgtaggttttcaaggggtacacttgacctgcacagcgatctcctttgcttttatagactttgtgttttttccttgtggataatgcattttttaccaactattgtttcacactgtaacaaaactatatttaaggtattaacacaatgcatttgttttgccttcaccaggcatggatatgcaaggtggcagtatatatcagatgacagagagaatgggctttttgaggctgcacgacgagagcttcatctcccttcggttaatgaaataattggtgctcagttgaacgaggcaaatgttagcatgtgctcactatgtcccttcctccaaattttgaagtgttgcatttcttatttcttccattccttttctaggggaatttggaaggtgcacaggaaggccaagcgaacacaacaagcatgtcgcattacaaggagacccagagaaagatagttgagttcttgagaaagagatatcatcttatggagagagccttgaatatggaatatgctgtggtacggtactaggcttttttccctgaacaggcatgcactcaaagctcacatgggagtgcacagtagctgttcatgtagtatcttgcagcattttttaaagcgtacctttttctcttgcagataaagaaaaaaattcctgttcctgatgatattactgaacaaggtgttccagcaggacatgctccttttattccagatatcagtgaactgttgcgggaattgcccaatcttgagccaatttgtaagtgatctcttattccctatatatatattccttctctccacctatgcaggaagagacagtcacatagtggcatagaacatgctggggcagatgctcacaatttttaatcatgtgtcatgcatgactcttgtcttactaaactctctctaaagatgtgttgttgttgacatatactcttgagtcttgaggtttgttcatttgtttgttttgataactgggatcctcgtttccagctaccaatgaattggtttctgagggcacagctggtcagttacaagttccccatctctacaataaggtgtgtaagcgcaatggcacacgttttcatgggtcatgggcgaacgcatcatttgttttcctcgagttaacattataaccagactgtgtacgtatgctctgcagatgtgtggagtgcttgaagagagtggtgcttatgcgctcagttccttctttggagacaagtccgcatcttctagtttggccaatagccttcgacagtttgaaactgtgtgcgggaatgtcgtcgaggccttgcgaccacaccaaaatggtactggcagtgccatcaaagaggaattggtagatgcagccaccaaagcagcagcagcagcagctcctcaacaagattcaggccatgatgcaccgcatgggcagtcttcgacagccaaggcggacatggaaatcgatggttgatttgtaggttccagagtggtaagaaagggaatccccctctaatcattatgtatactgtggtcagaatgtgcgctatatattgtaacatcaaagaaagcacctccaggcctgagggtgttactgctaatgcgtttggtttacttgttgtccttgtaatatgcatacacatttagaactcatgcagccattttgtgtgctcgaatncggtggatcgctgccctgttgtcttgtactgtgtttaagggcctgtttggnatacgaatggttaattataagacggggctaaagataagtacggattaactnatagttggctagcttgttgggtaagaaattacaaaatagtttgcaaaacaaaacatgttggtgcaagcgtggtcccaaaatgttaaaaacgaagaaacgatccatgcatatcttgtaagtatttacattggctcaattccaagcaacctttgcacttacattatacaaactagttcaattatgcatttctatacttgctttggtttgtgttggcatcaatcaccaaaaagggggagattgaaagggaattaggcttacacctatagtccctaattaattttggtggttgaattgcccaacacaaataattggactaactaatttgcccaagtgtatagaatatacaggtgtaaaaggttcacactcagccaataaaaagatcaagttttggattcaacaaaggagcaaagagacaaccgaaggcacctctggtctgggggcaccggactgtccggtgcaccagaggactcaaactcaaacttgccaccttcgggaattttcaaaggcactccgctataattcaccggactgtccggtgtacaccggacagtgtccggtgctccaaggaagagcggcctctggaactcgccagcctcgggaaaacgcagcggctgctccgctataattcaccggactgtccggtgtacaccggactgtccggtgaaccagcagagcaatggctacttcacgccaacggtcacc

SEQ ID NO: 42 - DNA, 4551 bp, Zea mays
  CDS (343)...(4332)
tgccccctctcctctcctctcgctcgtgcagaggaggagacagactccccccgtcgccgccctcgcgcctcctccgcccgcagctcaagccgagcgtcaccccgtcggctaccttccattctcgccgcgcttctcccactgggcacctcgcgccctcgtgacggtataaatagcccctagtcactcctcggcgctcctcaaacccaccaaaccctagcccacttagcggcggcggcgctagaggtggaacgagaccttggcggaggtagcgagggtagcgctgctcgctcgctcgttgggtttccatcccctactgcctagagcgagctctacgcgacgatgagcagccttgtcgagcggctgcgcgtgcggtcggagaggcggccgcggtacgcgctcgacgagtccgacgacgacctcccgctgcgcgttggggccggaaaggggaaggatcaacagaacgacgcgcccgccgagcggatcgagcgcgaggacgcgaaagaagaagcttgccagcgctgtggaaaaagtgataatctagtctcttgttcaacatgtacatacaaatttcacagaaaatgcttggttccttgcttaaacatcacatctgataaatggagctgcccagaatgtgtaagtccattaacatatatggagaggattctagatattgaagtgctggaagcacctcgtgaagattctagttccacagagcctcgatcaaagaagatggagcgatatcttatcaagtggaaaggattatcatacattcactgctcttgggtttcagaaaaagaatattcagaagccgcgaatatacaccctcgtctgaggactaggttgaataacttcagaaggcaaaaggaagccatgaaaatagaagcagaaagatctggtgaggacatcgttgcaattagaccggagtggacaactgttgacaggatccttgctagcagaaaaaacttagttggcgatcgggaatactatgttaaatggaatgaacttacatatgaggaatgtacatgggaaaatgagtctgacatcacggtgttccaacctgagattgaacgcttcaatgagatccagttcaggcgtaagaaatctggtgacaagggcaaggccactcgggagccacgccaattcaaggagagccctacgtttctttctggtggcacactacatccctatcagcttgaagggttaaactttttgcgatattcgtggtttcataacaaacgcgtaatccttggtgatgagatgggtcttgggaaaacgatacaaagtattgcttttcttgcctcactctttgaagacaagtttggtccgcatctggtcgttgctcccctctcaaccctgcggaattgggagcgtgaatttgcaacttgggcacctcaaatgaatgttgtaatgtattttggagctgctgcttctcgtgacattattaggaagtacgagttttactacccaaaagagaaactgaagaagctgaagaaaaagaaatcttctccatctaatgaagataagaagcagtcaaggataagatttgatgtcttattgacgtcttatgagatgatcaacatggactcgtctattctaaaaaatatagaatgggagtgcttggttgtggatgaggggcatcggttgaaaaacaaagattccaagttgtttggtcaacttaaagattataataccaaacatcgtgttctattaacggggaccccagtccagaataatcttgatgagcttttcatgcttatgcacttccttgagggtgaaagttttgggagtataactgatctccaagaagagttcaaggatataaaccaagacaagcaaattgagaagcttcatggaatgctgaagccacatcttcttcgaagattcaagaaggatgttatgaaagaacttcctccaaaaaaggaattgattctacgagttgaattgacaagaaaacagaaggagtactataaggcaattctcaccaagaattatgaagtgttagcccgtcgtaatggtggacatacatctctaataaacgttgtaatggagttgcgcaaactctgttgccatggattcatgattgatgaacctgatcttgaacctgccaatccagaagaaggtttaaggaggcttctagattcatcaggtaagatgcagctgctggacaagatgatggtgaaactgaaagagcagggtcatagagttctaatttattcacagttccagcacatgttggacttgctggaggactatttaagttacaggaaatggacttatgaacgcatcgatggcaagataagtggcgctgataggcagatacggatagatcgcttcaacgctaagaattcaactaggttttgctttcttctttctactagagctggtggtctgggaataaacttggcaactgcagatactgtaatcatctatgacagtgattggaacccacatgcggatttgcaagctatggcaagagctcatcgcttaggacagactagtaaggtgatgatatacaggcttgttagccgaggtacaattgaggagcgaatgatgcagcttacaaaaaagaagattttattggagcacttagttgttggtcgactcaccaaagctaataatgtcaatcaggaggagttggatgatattatacgctatggatcaaaggagctttttgaagacgagaatgatgaatctcgccaaattcattatgacgaagctgcaattgagaggttgttagaccgtgatcaagttgacggtgatgaatctgtggaagatgaagaagaagatggattcttaaaaggattcaaggttgcaaactttgaatatatcgatgaggcaaaggctcaggcagaaaaagaggaggcacggagaaaggctgcagctgaggctgaaaattctgaaagaaactactgggatgaactattgaaggatagatatgatgtacagaaagttgaagaacatactgctatgggaaaagggaaaagaagccgcaaacagatggctgccgctgatgaagatgacattcatgatttaagttccgaagatgaggattactcattggaggatgacatttcagataatgacacaagtttgcaaggaaatatttctgggaagaggggacaatattctaagagaaaatcacgtaatgttgattctattccattgatggagggcgaaggacgtaccttgagagttcttggattcaaccatgctcaacgagcaatgttcctacagacactcaatagattcggttttcagaattatgactggaaagagtatcttcctcgtcttaaaggaaaaagtgtcgaggaaatccagagatatgctgaacttgtcatggcacatcttgttgaagaaattaatgattctgactatttttcagatggcgttccaaaggaaatgatgcgtgttgatgatgtactagtcaggatagcaaacatatcccttatcgaggagaagatggctgccacaggaccaggaaaaattacaaacatttttcctaattacttgctctatgagttccaaggcttatctggtggaagaatatggaaagcggagcatgatctactgttactgagaggcatactgaagcatggatatgcaaggtggcagtatatatcagatgacagagagaatgggctttttgaggctgcacgacgagagctgcatctcccttcggttaatgaaataattggtgctcagttgaacgaggcaaatgggaatttggaaggtgcacaggaaggccaagcgaacacaacaagcatgtcgcattacaaggagatccagagaaagatagttgagttcttgagaaagagatatcatcttatggagagagccttgaatctggaatatgctgtgataaagaaaaaaattcctgttcctgatgatattactgaacaaggtgttccagcaggacatgctccgcttattccagatatcagtgaactgttgcgggaattgcccaatcttgagccaatttctaccaatgaattgatttctgagggcacagctggtcagttacaagttccccatctctacaataagatgtgtggagtgcttgaagagagtggtgcttatgcgctcagttccttctttggagacaagtccgcatcttctactttggccaatagccttcgacagtttgaaactgtgtgtgagaatgtcgtcgaggccttacgaccacaccaaaatggtactgccagtgccatcaaagaggaattggtagatgcagccaccaaagcagcagcagcagcagctcctcaacaagattcaggccatgatgcaccgcatgggcagtcttcgacagccaaggcggacatggaaatcgatggttgatttgtaggttccagagtggcaagaaagggaatcccctctaatcattatgtatactgtggtcagaatgtccgctatatattgtaacatcaaagaaagcacctccaggcctgagggtgttactgctaatgcgtttggtttacttgtccttgtaatatgcatacacatttagaactcatgcagccattgtgtgaaaaaaaaaaaaaaaaaaaaaaaaaa

SEQ ID NO: 43 - protein, 1358 aa, Zea mays
MSSLVERLRVRSERRPRYALDESDDDLPLRVGAGKGKDQQNDAPAERIEREDAKEEACQRCGKSDNLVSCSTCTYKFHRKCLVPCLNITSDKWSCPECVSPLTYMERILDIEVLEAPREDSSSTEPRSKKMERYLIKWKGLSYIHCSWVSEKEYSEAANIHPRLRTRLNNFRRQKEAMKIEAERSGEDIVAIRPEWTTVDRILASRKNLVGDREYYVKWNELTYEECTWENESDITVFQPEIERFNEIQFRRKKSGDKGKATREPRQFKESPTFLSGGTLHPYQLEGLNFLRYSWFHNKRVILGDEMGLGKTIQSIAFLASLFEDKFGPHLVVAPLSTLRNWEREFATWAPQMNVVMYFGAAASRDIIRKYEFYYPKEKLKKLKKKKSSPSNEDKKQSRIRFDVLLTSYEMINMDSSILKNIEWECLVVDEGHRLKNKDSKLFGQLKDYNTKHRVLLTGTPVQNNLDELFMLMHFLEGESFGSITDLQEEFKDINQDKQIEKLHGMLKPHLLRRFKKDVMKELPPKKELILRVELTRKQKEYYKAILTKNYEVLARRNGGHTSLINVVMELRKLCCHGFMIDEPDLEPANPEEGLRRLLDSSGKMQLLDKMMVKLKEQGHRVLIYSQFQHMLDLLEDYLSYRKWTYERIDGKISGADRQIRIDRFNAKNSTRFCFLLSTRAGGLGINLATADTVIIYDSDWNPHADLQAMARAHRLGQTSKVMIYRLVSRGTIEERMMQLTKKKILLEHLVVGRLTKANNVNQEELDDIIRYGSKELFEDENDESRQIHYDEAAIERLLDRDQVDGDESVEDEEEDGFLKGFKVANFEYIDEAKAQAEKEEARRKAAAEAENSERNYWDELLKDRYDVQKVEEHTAMGKGKRSRKQMAAADEDDIHDLSSEDEDYSLEDDISDNDTSLQGNISGKRGQYSKRKSRNVDSIPLMEGEGRTLRVLGFNHAQRAMFLQTLNRFGFQNYDWKEYLPRLKGKSVEEIQRYAELVMAHLVEEINDSDYFSDGVPKEMMRVDDVLVRIANISLIEEKMAATGPGKITNIFPNYLLYEFQGLSGGRIWKAEHDLLLLRGILKHGYARWQYISDDRENGLFEAARRELHLPSVNEIIGAQLNEANGNLEGAQEGQANTTSMSHYKEIQRKIVEFLRKRYHLMERALNLEYAVIKKKIPVPDDITEQGVPAGHAPLIPDISELLRELPNLEPISTNELISEGTAGQLQVPHLYNKMCGVLEESGAYALSSFFGDKSASSTLANSLRQFETVCENVVEALRPHQNGTASAIKEELVDAATKAAAAAAPQQDSGHDAPHGQSSTAKADMEIDGFVGSRVARKGIPSNHYVYCGQNVRYIL